(19) United States
(12) Patent Application Publication
(10) Pub. No.: US 2010/0137388 A1
(43) Pub. Date: Jun. 3, 2010

(54) Therapeutic Pro-Antibiotic Agents and Methods of Use Thereof

(76) Inventors: Christopher J. Barden, Halifax (CA); Annette L. Henneberry, Halifax (CA); David M. Byers, Halifax (CA); Christopher R. McMaster, Halifax (CA); Donald Weaver, Halifax (CA); Fan Wu, Halifax (CA)

Correspondence Address: ADE & COMPANY INC., 2157 Henderson Highway, WINNIPEG, MB, R2G1P9, CA

(21) Appl. No.: 12/595,841

(22) PCT Filed: Apr. 16, 2008
(86) PCT No.: PCT/CA2008/000632

§ 371 Date: Oct. 14, 2009

Related U.S. Application Data
(60) Provisional application No. 60/912,773, filed on Apr. 19, 2007.

(51) Int. Cl. A61K 31/41; C07D 257/04; A61P 31/12; A61P 31/04

(52) U.S. Cl. 514/382; 548/253

(57) ABSTRACT

The present invention provides for therapies characterized in part by co-administration or combination of antibiotic agents with medicinal compositions comprising as the pro-antibiotic active ingredient either a compound represented by a formula [I] or a pharmaceutically acceptable composite thereof; [wherein X represents oxygen, sulfur, NH, or N-alkyl; R₁ and R₂ represent hydrogen, tetrazole, or alkyltetrazole, respectively (with the restriction that if R₁ is not hydrogen, R₂ must be, and vice versa); R₃ represents e.g., benzyl, phenyl optionally substituted, biphenyl, napthyl, N-phenylcarboxamido, etc., R₄ represents e.g. carboxylic acid, or its alkyl esters, or a bioisoteric equivalent thereof, etc.; R₅ represents e.g. hydrogen, carboxylic acid, etc.] in particular, medicine which is useful as therapeutic and/or protective drugs for infectious and/or inflammatory diseases. Other relevant compounds are also provided.

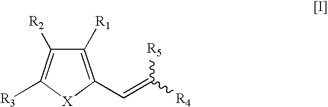

### PRIOR APPLICATION INFORMATION

The instant application claims the benefit of U.S. Provisional Patent Application 60/912,773, filed Apr. 19, 2007.

### BACKGROUND OF THE INVENTION

Infection is an invasion of a host organism by a foreign organism, generally to the detriment of normal function in the host. The deleterious effects of infection, often exacerbated by host response (i.e. inflammation), can be serious, even fatal. In treating humans and other animals for infection and post-infective inflammatory disease (e.g. septic shock), practitioners usually rely on chemical compounds known to have antibiotic affects, whether antiviral, antibacterial, antifungal, etc.

Unfortunately, many pathogens have become resistant to current antibiotic treatments. Antibiotic resistance is therefore an increasingly significant clinical issue, calling for novel antibiotics. Especially valuable would be new “pro-antibiotic” compounds, which can increase the potency, efficacy, and/or spectrum of activity of antibiotics when co-administered or given as combination therapy. Such compounds might or might not also be antibiotics (e.g. clavulanate).

Lipid A is the hydrophobic anchor of lipopolysaccharide (LPS) in the outer membrane of gram-negative bacteria. Lipid A is essential for growth of Escherichia coli as well as many other gram-negative pathogens. Many such pathogens are non-viable in the absence of LPS (Raetz and Whitfield, 2002, Annu Rev Biochem 71: 635-700; Wyckoff et al., 1998, Trends Microbiol 6: 154-159). Furthermore, it is known that decreased synthesis of lipid A can disrupt the integrity of the outer membrane, rendering bacteria more susceptible to other antibiotics (Lee et al, 2002, Acta Crystallogr D Biol Crystallogr 58: 864-6; Vuorio and Vaara, 1992, Antimicrob Agents Chemother 36:826-9). Accordingly, disrupting the synthesis of lipid A represents a fruitful strategy for novel antibiotics and pro-antibiotics. Since lipid A is toxic, such antibiotics or pro-antibiotics also have the potential to treat sick conditions associated with post-infective inflammatory diseases, such as systemic inflammatory response syndrome or septic shock.

All enzymes involved in E. coli lipid A biosynthesis have now been identified, and their structural genes have been cloned. The first step in lipid A biosynthesis is catalyzed by UDP-N-acetylglucosamine (UDP-GlcNAc) acyltransferase (LpxA), which transfers a β-hydroxy-fatty acyl group (typically 10-14 carbons in length, depending on the bacterial species) from acyl carrier protein (ACP) to the 3-OH glucosamine of UDP-GlcNAc (Wyckoff et al, 1998, J Biol Chem 273: 32369-72; Wyckoff and Raetz, 1999, J Biol Chem 274: 27047-55). Recently in WO/2006/092059, a series of antibiotic LpxA inhibitors have been discovered using a combination of rational drug design, chemical synthesis, and biological testing techniques. It has not been shown so far that such compounds have pro-antibiotic effects.

### DISCLOSURE OF THE INVENTION

As described above, it is understood that LpxA activity plays a major role in the progress of various infectious and post-infective inflammatory diseases. Thus, an object of the present invention is to provide medicinal compositions that are effective to remedy the infectious and/or inflammatory sick conditions and to cure or prevent the relevant disease(s). A second object of the present invention is to provide novel compounds to be used for the medicinal composition. As a result of the studies by the inventors of the present invention for aiming at achieving these objects, compounds not envisioned by WO/2006/092059 have been found to have antibiotic activity, thereby reaching the present invention. The present invention is also based in part on the significant discovery that a subset of these compounds can act as pro-antibiotics.

Accordingly, the present invention provides compounds of Formula I,

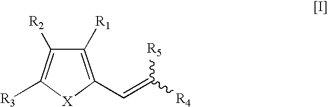

In Formula I, X is O, S, NH, or N-alkyl; R₁ and R₂ are each, independently, hydrogen, tetrazole, alkylcarboxytetrazole, or alkyltetrazole (with the restriction that if R₁ is not hydrogen, R₂ must be, and vice versa); R₃ is a halogen, alkyl halogen, 1,2-diphenylethyl, m-acrylylphenyl, substituted or unsubstituted N-phenylcarboxamido, substituted or unsubstituted alkyl, substituted or unsubstituted aryl or arylalkyl or substituted or unsubstituted heteroaryl or heteroarylalkyl; R₄ is carboxylic acid or alkylcarboxylic acid or their respective alkyl esters, or bioisosteric equivalents thereof; and R₅ is hydrogen, or carboxylic acid or its alkyl esters, or a bioisosteric equivalent thereof. R₄ may be connected cis or trans to the double bond, and R₅ will necessarily be in the cis/trans position opposite to that of R₄.

In particularly preferred embodiments, X is S; R₁ and R₂ are each, independently, hydrogen, tetrazole, 1-methyltetrazole, or 2-methyltetrazole (with the restriction that if R₁ is not hydrogen, R₂ must be, and vice versa); R₄ is carboxylic acid; R₅ is hydrogen; and R₃ is one of the following groups: substituted or unsubstituted benzyl; substituted or unsubstituted phenyl; substituted or unsubstituted biphenyl; substituted or unsubstituted biphenylmethyl; or substituted or unsubstituted naphthyl.

Bioisosteric equivalents are defined as those moieties which are structurally distinct yet functionally equivalent with respect to their biological effects. It is known in the art that certain moieties, although structurally distinct from each other, may behave identically within the biological milieu of the receptor microenvironment. Many bioisosteres are classical, replacing one atom in the moiety with another in the same chemical group in the periodic table, such as fluorine for chlorine or hydrogen (considered a surrogate member of Group 17). Other bioisosteres are non-classical, modifying all or substantially all of a moiety with structurally distinct molecular fragments that are nevertheless found to be biologically equivalent. For example, carboxylic acid can be substituted with phosphoric acid, sulfonic acid, tetrazole, or with some other moiety that carries a charge or that has the ability to donate electrons, such that their pharmacodynamic interactions are substantially similar resulting in an identical or near-identical biological effect. Such examples are not meant to be limiting. A novel bioisosteric substitution causes Formula II below to differ from Formula I by containing a functionalized ring moiety in place of Formula I's R₄ and R₅; such a substitution represents the approximate upper limit of size for bioisosteric equivalence.

In another aspect, the present invention provides compounds of Formula II,

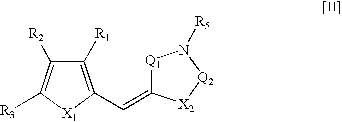

In Formula II, X₁ is O, S, NH, or N-alkyl; X₂ is C, O, S, NH, or N-alkyl; R₁ and R₂ are each, independently, hydrogen, tetrazole, or alkyltetrazole (with the restriction that if R₁ is not hydrogen, R₂ must be, and vice versa); R₃ is a halogen, alkylhalogen, substituted or unsubstituted N-phenylcarboxyamido, substituted or unsubstituted alkyl, substituted or unsubstituted aryl or arylalkyl or substituted or unsubstituted heteroaryl or heteroarylalkyl; R₅ is H or alkyl; and Q₁ and Q₂ are each, independently, C or C(O). If X₂ and Q₂ are both C, the bond between them may be single or double in order.

In compounds of Formulas I and II, when any of X, X₁, X₂, or R₁-R₅ is or is connected to an alkyl group, preferred alkyl groups are substituted or unsubstituted normal, branched, or cyclic C₁-C₆ alkyl groups. Particularly preferred alkyl groups are normal or branched C₁-C₄ alkyl groups. A substituted alkyl group includes at least one non-hydrogen substituent, such as an amino group, an alkylamino group or a dialkylamino group; a halogen, such as a fluoro, chloro, bromo, or iodo substituent; or hydroxyl. When R₃ is or is connected to a halogen, preferred groups include fluoro, chloro, bromo, and iodo. When R₃ is a substituted or unsubstituted aryl or heteroaryl group, preferred groups include substituted and unsubstituted phenyl, napththyl, indolyl, imidazolyl, and pyridyl. When R₃ is a substituted or unsubstituted arylalkyl or heteroarylalkyl group, preferred groups include substituted and unsubstituted benzyl, napththylmethyl, indolylmethyl, imidazolylmethyl, biphenylmethyl, and pyridylmethyl groups. Preferred substituents on aryl, heteroaryl, arylalkyl, heteroarylalkyl, and N-phenylcarboxyamido groups are independently selected from the group consisting of amino; alkyl-substituted amino; cyano; halogens, such as fluoro, chloro, bromo and iodo; mercapto; nitro; hydroxyl; or alkoxy and alkyl groups, preferably normal or branched C₁-C₆-alkyl or alkoxy groups, most preferably methyl or methoxy groups.

In a third aspect, the invention features pharmaceutical compositions comprising one or more of the antibiotic compounds of Formula I or II and a pharmaceutically acceptable carrier.

In a fourth aspect, the invention features pharmaceutical compositions comprising (a) one or more of the pro-antibiotic compounds of Formula I or II; (b) one or more of the antibiotic agent(s) known in the art; and (c) a pharmaceutically acceptable carrier.

In a fifth aspect, the invention features co-administration of one or more antibiotic agent(s) with a pharmaceutical composition comprising one or more of the pro-antibiotic compounds of Formula I or II and a pharmaceutically acceptable carrier.

In a sixth aspect, the invention features a method of treating and/or preventing an infectious disease (such as blood-stream infection, brucellosis, campylobacteriosis, Cat Scratch fever, cholera, gonorrhea, legionellosis, leptospirosis, Lyme disease, melioidosis, meningitis, pertussis, plague, pneumonia, salmonellosis, shigellosis, syphilis, tularemia, typhoid fever, urinary tract infection) in a subject. The method includes administering to the subject a therapeutically effective amount of one or more antibiotic compounds of Formula I or II.

In a seventh aspect, the invention features an additional method of treating and/or preventing an infectious disease in a subject. The method includes administering to the subject a therapeutically effective amount of one or more pro-antibiotic compounds of Formula I or II, and one or more antibiotic agent(s) known in the art.

In an eighth aspect, the invention features a method of treating and/or preventing a post-infective inflammatory disease (such as systemic inflammatory response syndrome or septic shock) in a subject. The method includes administering to the subject a therapeutically effective amount of one or more antibiotic compounds of Formula I or II.

In a ninth aspect, the invention features an additional method of treating and/or preventing a post-infective inflammatory disease in a subject. The method includes administering to the subject a therapeutically effective amount of one or more pro-antibiotic compounds of Formula I or II, and one or more antibiotic agent(s) known in the art.

The present invention also includes pharmaceutically acceptable composites of the antibiotic and pro-antibiotic compounds of the invention. A “pharmaceutically acceptable composite” includes a salt that retains the desired biological activity of the parent antibiotic or pro-antibiotic compound and does not impart any undesired toxicological effects. Examples of such salts are salts of acids such as hydrochloric acid, hydrobromic acid, sulfuric acid, phosphoric acid, nitric acid, and the like; acetic acid, oxalic acid, tartaric acid, succinic acid, malic acid, benzoic acid, pamoic acid, alginic acid, methanesulfonic acid, naphthalenesulfonic acid, and the like. Also included are salts of cations such as sodium, potassium, lithium, zinc, copper, barium, bismuth, calcium, and the like; or organic cations such as trialkylammonium. Combinations of the above salts are also useful.

It is of note that the compounds in the invention may be prepared to be administered in a variety of ways, for example, topically, orally, intravenously, intramuscularly, subcutaneously, intraperitoneally, intranasally or by local or systemic intravascular infusion using means known in the art and as discussed below.

It is of note that as discussed herein, the compounds in the invention may be arranged to be delivered at a concentration of about 1 μM to about 50 mM; or 10 μM to 50 mM; or 100 μM to 50 mM; or 1 mM to 50 mM. As will be appreciated by one of skill in the art, this may be the effective concentration, that is, a sufficient dosage is administered such that a concentration within one of the envisioned ranges is attained at the required site. As will be apparent to one knowledgeable in the art, the total dosage will vary according to many factors, including but by no means limited to the weight, age and condition of the individual or patient.

In some embodiments, one or more of the compounds in the invention may be combined at concentrations or dosages discussed above with a pharmaceutically or pharmacologically acceptable carrier, excipient or diluent, either biodegradable or non-biodegradable. Exemplary examples of carriers include, but are by no means limited to, for example, poly(ethylene-vinyl acetate), copolymers of lactic acid and glycolic acid, poly(lactic acid), gelatin, collagen matrices, polysaccharides, poly(D,L lactide), poly(malic acid), poly(caprolactone), celluloses, albumin, starch, casein, dextran, polyesters, ethanol, mathacrylate, polyurethane, polyethylene, vinyl polymers, glycols, mixtures thereof and the like. Standard excipients include gelatin, casein, lecithin, gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidol silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethycellulose phthalate, noncrystalline cellulose, magnesium aluminum silicate, triethanolamine, polyvinyl alcohol, polyvinylpyrrolidone, sugars and starches. See, for example, Remington: The Science and Practice of Pharmacy, 1995, Gennaro ed.

As will be apparent to one knowledgeable in the art, specific carriers and carrier combinations known in the art may be selected based on their properties and release characteristics in view of the intended use. Specifically, the carrier may be pH-sensitive, thermo-sensitive, thermo-gelling, arranged for sustained release or a quick burst. In some embodiments, carriers of different classes may be used in combination for multiple effects, for example, a quick burst followed by sustained release.

In other embodiments, one or more of the compounds in the invention at concentrations or dosages described above may be encapsulated for delivery. Specifically, the compounds may be encapsulated in biodegradable microspheres, microcapsules, microparticles, or nanospheres. The delivery vehicles may be composed of, for example, hyaluronic acid, polyethylene glycol, poly(lactic acid), gelatin, poly(E-caprolactone), or a poly(lactic-glycolic) acid polymer. Combinations may also be used, as, for example, gelatin nanospheres may be coated with a polymer of poly(lactic-glycolic) acid. As will be apparent to one knowledgeable in the art, these and other suitable delivery vehicles may be prepared according to protocols known in the art and utilized for delivery of the compounds.

It is of note that the compounds in the invention may be combined with permeation enhancers known in the art for improving delivery. Examples of permeation enhancers include, but are by no means limited to those compounds described in U.S. Pat. Nos. 3,472,931; 3,527,864; 3,896,238; 3,903,256; 3,952,099; 4,046,886; 4,130,643; 4,130,667; 4,299,826; 4,335,115; 4,343,798; 4,379,454; 4,405,616; 4,746,515; 4,788,062; 4,820,720; 4,863,738; 4,863,970; and 5,378,730; British Pat. No. 1,011,949; and Idson, 1975, J. Pharm. Sci. 64:901-924.

In one embodiment, the present invention provides a method of treating a subject suffering from a bacterial infection, such as an infection by Pseudomonas aeruginosa, by Helicobacter pylori, or by Klebsiella pneumoniae. The method comprises the step of administering to the subject a therapeutically effective amount of an antibiotic compound of the invention. The subject can be an individual who is suffering from, or susceptible to, infection by a bacterial organism, and who may or may not suffer from, or be susceptible to, systemic inflammatory response syndrome or septic shock.

In a further embodiment, the present invention provides an additional method of treating a subject suffering from a bacterial infection, such as an infection by Pseudomonas aeruginosa, by Helicobacter pylori, or by Klebsiella pneumoniae. The method comprises the step of administering to the subject a therapeutically effective amount of a pro-antibiotic compound of the invention, and an antibiotic known in the art. The subject can be an individual who is suffering from, or susceptible to, infection by a bacterial organism, and who may or may not suffer from, or be susceptible to, systemic inflammatory response syndrome or septic shock.

The use of the terms “antibiotic” and “pro-antibiotic” in describing the invention is not mutually exclusive. It is known that increasing the permeability of a pathogen's outer membrane causes non-viability at a certain threshold of activity. Compounds of Formula I or II that are pro-antibiotic at lower doses may be antibiotic at higher doses. Furthermore, reducing the quantity of lipid A produced by a pathogen may demonstrate anti-inflammatory effects at therapeutic doses lower than those required for antibiotic activity. These considerations will affect the dosing for the treatments disclosed above.

Several aspects of the invention as disclosed above provide for co-administration or combination therapy of a compound of the invention and one or more antibiotic agent(s) known in the art. In co-administration procedures, the agents may be administered concurrently or sequentially. In one embodiment, the pro-antibiotic compounds described herein are administered prior to the other active agent(s). The pharmaceutical formulations and modes of administration may be any of those described herein. In addition, the two or more co-administered agents may each be administered using different modes or different formulations.

Examples of such antibiotic agents include, but are not limited to, almecillin, amdinocillin, amikacin, amoxicillin, amphomycin, amphotericin B, ampicillin, azacitidine, azaserine, azithromycin, azlocillin, aztreonam, artemisinin, allopurinol, amicacin, aminoglycosides, amphotericin B, ampicillin, ansamycins, anthracyclines, antimycotics, azithromycin. bacampicillin, bacitracin, benzyl penicilloyl-polylysine, bleomycin, brefeldin A, butoconazole, candicidin, capreomycin, carbenicillin, cefaclor, cefadroxil, cefamandole, cefazoline, cefdinir, cefepime, cefixime, cefinenoxime, cefinetazole, cefodizime, cefonicid, cefoperazone, ceforanide, cefotaxime, cefotetan, cefotiam, cefoxitin, cefpiramide, cefpodoxime, cefprozil, cefsulodin, ceftazidime, ceftibuten, ceftizoxime, ceftriaxone, cefuroxime, cephacetrile, cephalexin, cephaloglycin, cephaloridine, cephalothin, cephapirin, cephradine, chloramphenicol, cilastatin, cinnamycin, ciprofloxacin, clarithromycin, clavulanic acid, clindamycin, clioquinol, cloxacillin, colistimethate, colistin, cyclacillin, cycloserine, cyclosporine, cyclo-(Leu-Pro), camptothecin, cefataxime, cephalexin, cephalosporins, chalcomycin, chartreusin, chlorotetracyclines, chlorothricin, chrymutasins, chrysomicin M, chrysomicin V, clomocyclines, dactinomycin, dalbavancin, dalfopristin, daptomycin, daunorubicin, demeclocycline, detorubicin, dicloxacillin, dihydrostreptomycin, dirithromycin, doxorubicin, doxycycline, DNM-113, DNM-123, DNM-131, DNM-141, DNM-142, DNM-145, ellipticines, elsamicin, epirubicin, erythromycin, eveminomycin, filipins, fluconazoles, fungichromins, fusidic acid, floxacillin, fosfomycin, gentamycin, gilvocarin, griseofulvin, griseoviridin, guamecyclines, gemifloxacin, gramicidin, hetacillin, idarubicin, imipenem, iseganan, ivermectin, ilosamides, itraconazoles, kanamycin, laspartomycin, linezolid, loracarbef, L-161,240, L-159,692, L-573,655, lankamycin, lincomycin, magainin, meclocycline, meropenem, methacycline, mezlocillin, minocycline, mitomycin, moenomycin, moxalactam, moxifloxacin, mycophenolic acid, macrolides, methicillins, mitoxantrone, nafcillin, natamycin, neomycin, netilmicin, niphimycin, nitrofurantoin, novobiocin, nalidixic acid, norfloxin, nystatin, nystatins, ofloxacin, oleanomycin, oxytetracyline, paromomycin, penicillamine, phenethicillin, piperacillin, plicamycin, pristinamycin, pecilocin, penicillins, pesticides, phosphomycin, pimarcin, platensimycin, polyenes, polymyxin B, polymyxin E, quinupristin, quinolones, ravidomycin, reserpines, rifamycin, ristocetins A and B, rifabutin, rifampin, rifamycin, rolitetracycline, sisomycin, spiramycin, spironolactone, sulfacetamide sodium, sulphonamide, spectrinomycin, streptomycin, streptozocin, sulbactam, sultamicillin, tacrolimus, tazobactam, teicoplanin, telithromycin, teramycins, tetracyclines, thiamphenicols, thiolutins, tobramycin, tyrothricin, ticarcillin, tigecycline, tobramycin, troleandomycin, tunicamycin, tyrthricin, vancomycin, vidarabine, viomycin, virginiamycin, and wortmannins; the presence of a plural item in the foregoing list meaning to refer to one or more members of a family of antibiotics known in the art by that name. Which agent or agents should be co-administered or compounded in combination with compounds of the present invention depends on a number of factors, including but not necessarily limited to the efficacy of the agent or agents in the absence of pro-antibiotic compounds, the mechanism of action of those agent or agents, the identity of the pathogen causing or potentiating the sick condition, and/or the severity of the sick condition in the subject.

### EMBODIMENTS FOR CARRYING OUT THE INVENTION

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

As used herein, “effective amount” refers to the administration of an amount of a given compound that achieves the desired effect.

As used herein, “purified” does not require absolute purity but is instead intended as a relative definition. For example, purification of starting material or natural material to at least one order of magnitude, preferably two or three orders of magnitude is expressly contemplated as falling within the definition of “purified”.

As used herein, the term “isolated” requires that the material be removed from its original environment.

As used herein, the term “treating” in its various grammatical forms refers to preventing, curing, reversing, attenuating, alleviating, minimizing, suppressing or halting the deleterious effects of a disease state, disease progression, disease causative agent or other abnormal condition.

The following compounds of Formula I are preferred embodiments of the invention: (Z)-methyl 3-(5-benzyl-4-(1H-tetrazol-5-yl)thiophen-2-yl)acrylate, (E)-methyl 3-(5-benzyl-4-(1H-tetrazol-5-yl)thiophen-2-yl)acrylate, (Z)-3-(5-benzyl-4-(1H-tetrazol-5-yl)thiophen-2-yl)acrylic acid, (E)-3-(5-benzyl-4-(1H-tetrazol-5-yl)thiophen-2-yl)acrylic acid, (E)-methyl 3-(5-benzyl-4-)1-methyl-1H-tetrazol-5-yl)thiophen-2-yl)acrylate, (E)-methyl 3-(5-benzyl-4-(2-methyl-2H-tetrazol-5-yl)thiophen-2-yl)acrylate, (E)-3-(5-benzyl-4-(2-methyl-2H-tetrazol-5-yl)thiophen-2-yl)acrylic acid, (E)-3-(5-benzyl-4-(1-methyl-1H-tetrazol-5-yl)thiophen-2-yl)acrylic acid, (E)-3-(5-(4-fluorophenyl)-4-(1H-tetrazol-5-yl)thiophen-2-yl)acrylic acid, (E)-3-(5-(4-biphenylyl)-4-(1H-tetrazol-5-yl)thiophen-2-yl)acrylic acid, (E)-methyl 3-(5-(naphthalen-1-yl)-4-(1H-tetrazol-5-yl)thiophen-2-yl)acrylate, (E)-3-(5-(naphthalen-1-yl)-4-(1H-tetrazol-5-yl)thiophen-2-yl)acrylic acid, (E)-methyl 3-(5-(4-fluorophenyl)-3-(1H-tetrazol-5-yl)thiophen-2-yl)acrylate, (E)-3-(5-(4-fluorophenyl)-3-(1H-tetrazol-5-yl)thiophen-2-yl)acrylic acid, (E)-methyl 3-(5-(4-fluorophenyl)-3-(2-methyl-2H-tetrazol-5-yl)thiophen-2-yl)acrylate, (E)-3-(5-(4-fluorophenyl)-3-(2-methyl-2H-tetrazol-5-yl)thiophen-2-yl)acrylic acid, (E)-3-(5-(4-fluorophenyl)-3-(1-methyl-1H-tetrazol-5-yl)thiophen-2-yl)acrylic acid, (E)-methyl 3-(4-(2-methyl-2H-tetrazol-5-yl)-5-(naphthalen-1-yl)thiophen-2-yl)acrylate, (E)-methyl 3-(5-(4-biphenyl)-3-(1H-tetrazol-5-yl)thiophen-2-yl)acrylate, (E)-3-(5-(biphenyl-4-yl)-3-(1H-tetrazol-5-yl)thiophen-2-yl)acrylic acid, (E)-methyl 3-(5-benzyl-3-(1H-tetrazol-5-yl)thiophen-2-yl)acrylate, 2-((5-benzyl-4-(1H-tetrazol-5-yl)thiophen-2-yl)methylene)malonic acid, (E)-3-(5-benzyl-4-(2-(2-carboxyethyl)-2H-tetrazol-5-yl)thiophen-2-yl)acrylic acid, and (E)-3-(3-((5-((E)-2-carboxyvinyl)-3-(1H-tetrazol-5-yl)thiophen-2-yl)methyl)phenyl)acrylic acid.

The following compounds of Formula II are preferred embodiments of the invention: 5-((5-benzyl-3-(1H-tetrazol-5-yl)thiophen-2-yl)methylene)thiazolidine-2,4-dione.

The representative compounds usable in the present invention including the compounds prepared in the Examples described below are presented in Table 1 and 2. The abbreviations and the reference symbols in the tables have the following meanings, respectively: Me: methyl, Et: ethyl, Naph: naphthyl, Ph: phenyl, Bn: benzyl, Biph: biphenyl; Tet: tetrazole; 1MeTet: 1-methyltetrazole; 2MeTet: 2-methyltetrazole. R₄ is attached trans unless otherwise noted. Furthermore, it is noted that the names for these compounds are provided in the list above, as well as below in the synthetic Examples alongside their ‘DNM’ designation in parentheses. It is further noted that the chemical names of these compounds would be obvious to one of skill in the art on reviewing Table 1 and/or 2. Accordingly, it is to be understood that the ‘DNM’ numbers used herein may be used interchangeably with the corresponding structure shown in Table 1 and/or 2 or the proper chemical name, which as discussed above is either provided below or may be deduced from the structure using standard nomenclature rules known in the art.

TABLE I

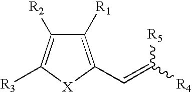

| Compound | | | | | | |
|---|---|---|---|---|---|---|
| designation | X | R₁ | R₂ | R₃ | R₄ | R₅ |
| DNM0194 | S | H | Tet | Benzyl | —COOMe | H |
| DNM0195 | S | H | Tet | Benzyl | —COOH | H |
| DNM0196 | S | H | 1MeTet | Benzyl | —COOH | H |
| DNM0197 | S | H | 2MeTet | Benzyl | —COOH | H |
| DNM0224 | S | H | Tet | p-fluorophenyl | —COOH | H |
| DNM0225 | S | H | Tet | Biph | —COOH | H |
| DNM0226 | S | H | 2MeTet | Benzyl | —COOMe | H |
| DNM0227 | S | H | 1MeTet | Benzyl | —COOMe | H |
| DNM0229 | S | Tet | H | p-fluorophenyl | —COOMe | H |
| DNM0230 | S | Tet | H | p-fluorophenyl | —COOH | H |
| DNM0231 | S | 2MeTet | H | p-fluorophenyl | —COOMe | H |
| DNM0232 | S | 2MeTet | H | p-fluorophenyl | —COOH | H |
| DNM0233 | S | 1MeTet | H | p-fluorophenyl | —COOH | H |
| DNM0234 | S | H | Tet | Naph | —COOMe | H |
| DNM0235 | S | H | Tet | Naph | —COOH | H |
| DNM0236 | S | H | 2MeTet | Naph | —COOMe | H |
| DNM0238 | S | H | Tet | 1,2-diphenylethyl | —COOMe | H |
| DNM0239 | S | H | Tet | 1,2-diphenylethyl | —COOH | H |
| DNM0240 | S | Tet | H | Biph | —COOMe | H |
| DNM0243 | S | Tet | H | Benzyl | —COOMe | H |
| DNM0245 | S | Tet | H | Biph | —COOH | H |
| DNM0252 | S | H | 3-N-ethylcarboxy Tet | Benzyl | —COOH | H |
| DNM0264 | S | H | Tet | Benzyl | —COOH | —COOH |
| DNM0270 | S | H | Tet | 3-acrylylphenyl | —COOH | H |

TABLE 2

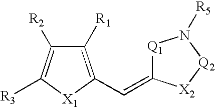

| Compound | | | | | | | | |
|---|---|---|---|---|---|---|---|---|
| designation | X₁ | X₂ | Q₁ | Q₂ | R₁ | R₂ | R₃ | R₅ |
| DNM0246 | S | S | C═O | C═O | Tet | H | Benzyl | H |

### Rational Drug Design of LpxA Inhibitors

The x-ray structure of the E. coli and Helicobacter pylori enzymes have been determined and are trimers of identical 30 kDa subunits. Chemical modification studies have indicated that the active site of LpxA is in a cleft shared by two adjacent subunits (Lee et al, 2002, Acta Crystallogr D Biol Cystallogr 58:864-6). A three-point pharmacophore can therefore be surmised that blocks the channel produced by these subunits (WO/2006/092059). A set of active LpxA inhibitors was analyzed using three-dimensional descriptors that quantify the binding energy of such inhibitors in various orientations around the pharmacophore. Quantitative Structure-Activity Relationship techniques were then applied to these data to afford a statistical Partial Least Squares model for prediction of activity. Using this model, compounds of formula I were predicted to be enriched in active LpxA inhibitors.

### Assays for Detecting the Activity of LpxA Inhibitors

Measurement of LpxA activity was based on increased mobility of radiolabeled UDP-N-acetyl-D-glucosamine on thin layer chromatography (TLC) plates upon acylation. One μl of test compound of appropriate dilution in ethanol was mixed with 2.5 μM β-hydroxy-myristoyl-ACP and 0.5 μM E. coli LpxA in 1% bovine serum albumin and 40 mM Na-Hepes (pH 8.0) at room temperature (total volume 8 μl). The reaction was initiated by adding 2 μl of UDP-[³H]N-acetyl-D-glucosamine and aliquots were removed at various time intervals and spotted directly on silica gel G plates. The LpxA assay was validated based on its specificity for β-hydroxymyristoyl-ACP.

### Assays for Detecting Antibiotic Activity

Susceptibilities to the compounds were determined using the National Committee for Clinical Laboratory Standards (NCCLS) M7-A6 broth microdilution method. Cation-adjusted Mueller-Hinton broth (Ca²⁺, 25 μg/mL; Mg²⁺, 12.5 μg/mL) microdilution panels were prepared to contain antimicrobial doubling dilution concentrations of an appropriate range. DMSO (dimethylsulfoxide) controls were incorporated into the panel to mimic the quantity of DMSO used in dissolving some of the compounds at the higher concentrations. Each final panel well volume was 100 μL with a bacterial inoculum of 5×10⁵ CFU (colony forming units)/mL. Panels were read following 16 to 20 h of incubation at 35 degrees Celsius in ambient air. The MIC (minimum inhibitory concentration) was defined as the lowest concentration of antimicrobial inhibiting visible growth.

### Synthetic Methods

Compounds of the invention can be prepared using one or more of the following general methods.

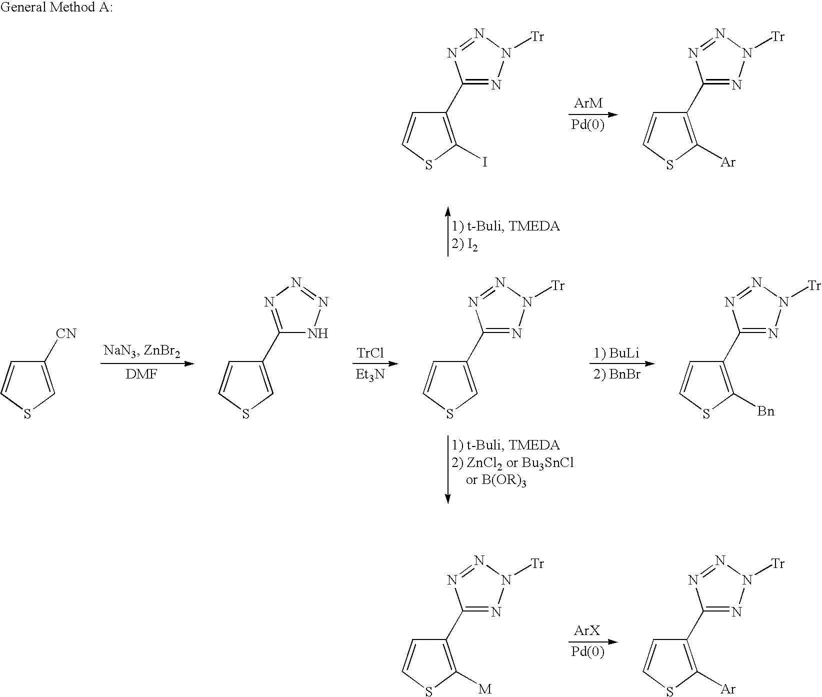

Method A was also used to prepare 5-(2-aryl-thiophen-3-yl)tetrazole analogues. The commercially available thiophene-3 -carbonitrile was converted to 5-(thiophen-3-yl)-1H-tetrazole by refluxing in DMF with sodium azide and ZnBr₂. The resultant 3-(5-tetrazolyl)thiophene was protected by trityl group, and then lithiated at position 2 with BuLi. The lithiated compound was further converted to 2-aryl substituted 3-(5-tetrazolyl)thiophene.

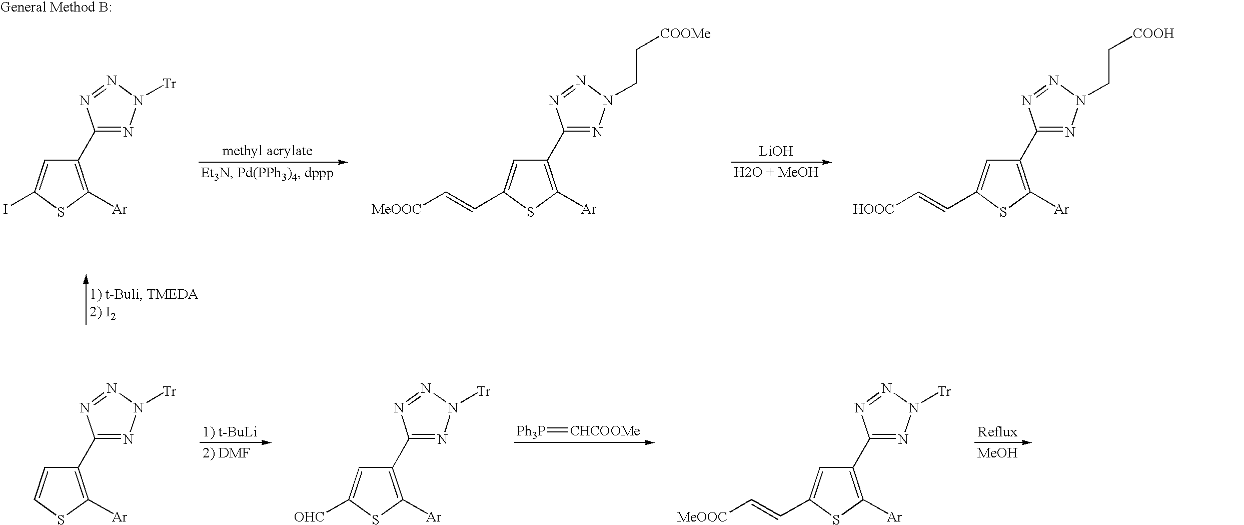

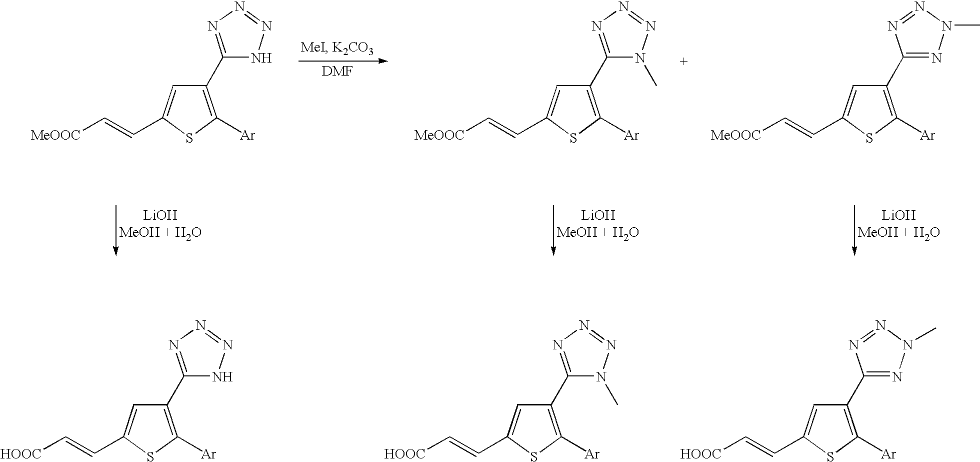

Method B was used to synthesize (E)-3-(5-aryl-4-(tetrazol-5-yl)thiophen-2-yl)acrylate analogues. 2-aryl-3-(5-tetrazolyl)thiophene was further lithiated at position 5 with t-BuLi and TMEDA. The lithium species was quenched with DMF to afford 2-aryl-5-formyl-3-(5-tetrazolyl)thiophene. The resulting aldehyde was transferred into acrylate moiety by Wittig reaction.

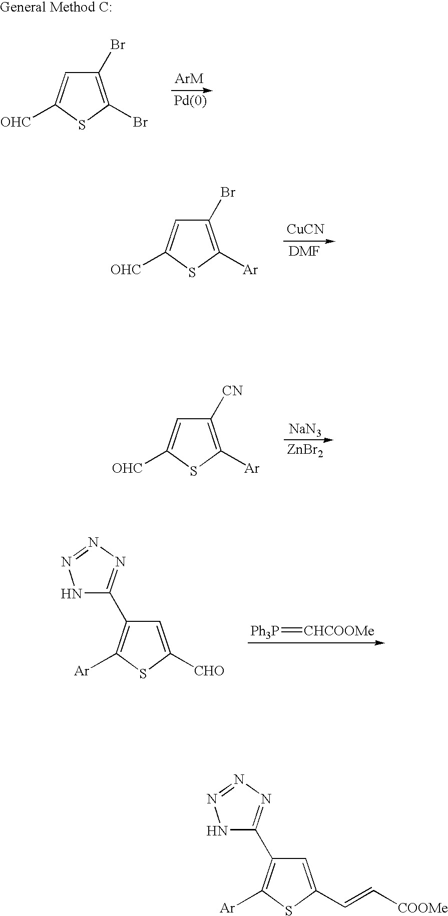

Method C was used to prepare (E)-3-(5-aryl-4-(tetrazol-5-yl)thiophen-2-yl)acrylate analogues. Selective Pd catalyzed cross-coupling of 4,5-dibromothiophene-2-carbaldehyde at position 5 using organometallic reagents gave 4-bromo-4-aryl-thiophene-2-carbaldehyde, which was further transferred to nitrile with CuCN. The nitrile was treated with sodium azide with the existence of zinc bromide to furnish the tetrazole moiety. The Wittig reaction of the aldehyde afforded (E)-3-(5-aryl-4-(tetrazol-5-yl)thiophen-2-yl)acrylate.

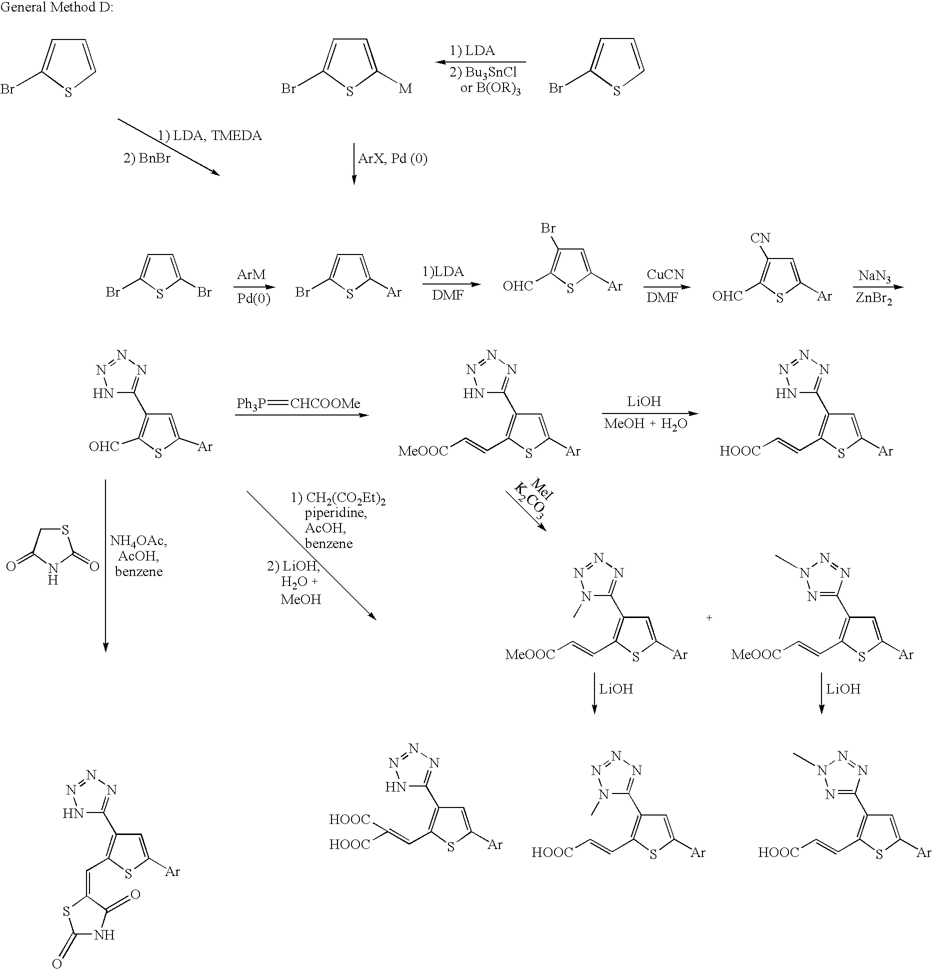

Method D was used for the synthesis of (E)-3-(5-aryl-3-(tetrazol-5-yl)thiophen-2-yl)acrylate analogues. The mono cross-coupling of 2,5-dibromothiophene with organometallic reagent such as zinc, tin and boric acid afforded 5-aryl-2-bromothiophene. Lithiation with LDA followed by quenching with DMF gave 5-aryl-3-bromothiophene-2-carbaldehyde, in which Br-3 was further transferred to nitrile. The nitrile was treated with sodium azide and zinc bromide to afford the tetrazole moiety. The Wittig reaction of 5-aryl-3-tetrazol-5-yl)thiophene-2-carbaldehyde gave (E)-3-(5-aryl-3-(tetrazol-5-yl)thiophen-2-yl)acrylate.

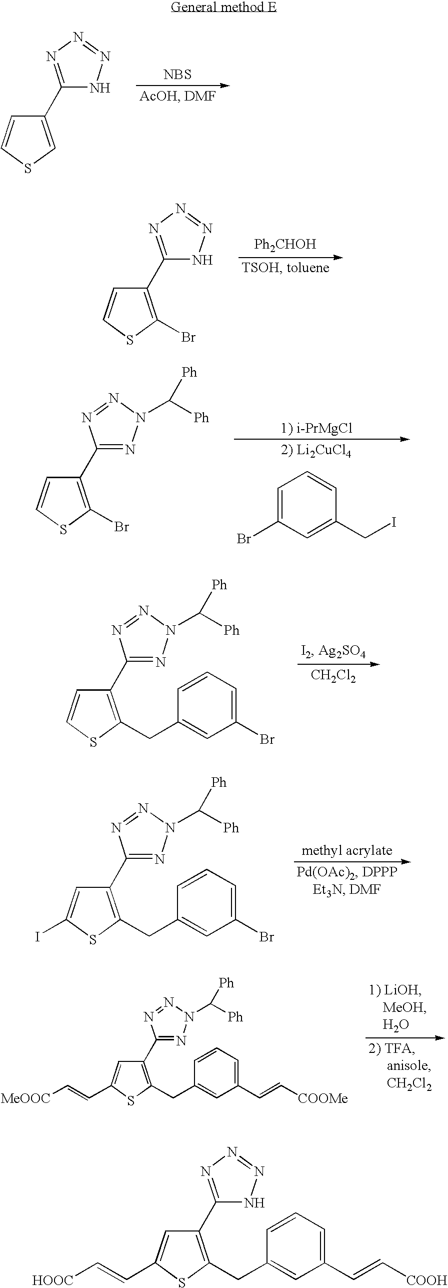

Method E was used to introduce carboxyvinylbenzyl substitutent to the thiophene ring. 5-(Thiophen-3-yl)-1H-tetrazole was brominated region-selectively at C₂ position of thiophene ring. The introduction of benzyl group was smoothly carried out by tetrachlorocuprate(II)-catalyzed coupling reaction of thienylmagnesium reagents and bromobenzyliodides. The Heck reaction of the bromobenzyl group and acrylate furnished the introduction of carboxyvinylbenzyl substitutent.

This invention is further illustrated by the following examples that should not be construed as limiting.

### Examples

### Example 1

### Synthesis of DNM0194

Methods A and B or Method C were Used.

5-(Thiophen-3-yl)-1H-tetrazole A mixture of thiophene-3-carbonitrile (5.72 g, 52.4 mmol), sodium azide (6.83 g, 105.1 mmol) and zinc bromide (23.65 g, 105.0 mmol) in 50 mL of dry DMF was heated to reflux and monitored by TLC until the reaction was complete (˜5 h). After being cooled to room temperature, 150 mL of 1 N aqueous HCl was added to precipitate the crude product. The white solid product was collected, washed with water and ether, and dried together with phosphorous pentaoxide under vacuum. 7.80 g (98%) of product was obtained as a white solid, mp: 203.0-205.0° C. [lit. mp: 244.8-255.3° C., Elpern, B.; Nachod, F. C. J. Am. Chem. Soc. 1950, 72, 3379-3382].

5-(Thiophen-3-yl)-2-trityl-2H-tetrazole 5-(Thiophen-3-yl)-1H-tetrazole (3.04 g, 20.0 mmol) was suspended in 40 mL of THF. Triethylamine (3.1 mL, 22 mmol) was added. After stirring for 10 minutes at room temperature, the reaction became a clear solution. Trityl chloride (6.13 g, 22.0 mmol) was added. The reaction was monitored with TLC until complete (˜1 h). The solution was diluted with 100 mL of ethyl acetate and filtered. The filtrate was washed with H₂O and brine, dried over anhydrous sodium sulfate, and concentrated. The residue was suspended in 50 mL of ether, well stirred for 1 h, and filtered. The white solid was collected and dried under vacuum. 7.65 g (97%) of product was obtained as a white solid, ¹H NMR (CDCl₃, 500 MHz) δ 8.05 (dd, J₁=1.14 Hz, J₂=2.98 Hz, 1H), 7.69 (dd, J₁=1.14 Hz, J₂=5.05 Hz, 1H), 7.38 (dd, J₁=3.01 Hz, J₂=5.05 Hz, 1H), 7.33 (m, 9H), 7.16 (m, 6H); ¹³C NMR (CDCl₃, 125 MHz) δ 160.78, 141.43, 130.34, 129.13, 128.35, 127.81, 126.59, 126.51, 125.54, 83.10.

5-(2-Benzylthiophen-3-yl)-2-trityl-2H-tetrazole A solution of 5-(Thiophen-3-yl)-2-trityl-2H-tetrazole (7.88 g, 20 mmol) in 100 mL of THF was cooled in a dry ice/acetone bath. To the solution was added a solution of n-BuLi (10 mL, 2.5 M, 25 mmol) dropwise. After addition was complete, the solution was stirred for 30 min at −78° C. Benzyl bromide (3.6 mL, 30.3 mmol) was added. The reaction was stirred for 30 min at −78° C., then warmed to room temperature, stirred at room temperature until complete, quenched with saturated NH₄Cl, extracted with EtOAc. The organic phase was dried over anhydrous MgSO₄ and concentrated. The residue was purified by flash chromatography (hexane: CH₂Cl₂: diethyl ether=200:100:9). 8.3 g (85%) of product was obtained as a white solid, ¹H NMR (CDCl₃, 500 MHz) δ 7.65 (d, J=5.34 Hz, 1H), 7.31 (m, 9H), 7.17 (m, 4H), 7.12 (m, 6H), 7.08 (m, 2H); ¹³C NMR (CDCl₃, 125 MHz) δ 160.70, 144.99, 141.38, 139.88, 130.29, 128.70, 128.34, 128.28, 127.96, 127.73, 126.31, 124.55, 123.58, 83.07, 34.82.

5-Benzyl-4-(2-trityl-2H-tetrazol-5-yl)thiophene-2-carbaldehyde A solution of 5-(2-benzylthiophen-3-yl)-2-trityl-2H-tetrazole (14.98 g, 30.95 mmol) and TMEDA (5.1 mL, 34.0 mmol) in 150 mL of THF was cooled in a dry ice/acetone bath. A solution of t-BuLi (20.0 mL, 1.7 M, 34.0 mmol) was added dropwise. After addition was complete, the solution was stirred for 1 h at −78° C. To the solution was added DMF (1.2 mL, 15 mmol). The reaction was stirred for 15 min at −78° C., then removed the cooling bath and warmed to room temperature naturally. The reaction was stirred at room temperature until complete, quenched with saturated NH₄Cl, extracted with EtOAc. The organic phase was dried over anhydrous MgSO₄ and concentrated. The residue was purified by recrystallization to afford 10.5 g of product. The mother liquid was further purified by flash chromatography (hexane:CH₂Cl₂:diethyl ether=100:100:6) to give 2.3 g of product, which brought a total yield of 12.8 g (81%) of product as a white solid.

(Z) and (E)-methyl 3-(5-benzyl-4-(2-trityl-2H-tetrazol-5-yl)thiophen-2-yl)acrylate A solution of 5-benzyl-4-(2-trityl-2H-tetrazol-5-yl)thiophene-2-carbaldehyde (1.75 g, 3.42 mmol) and Ph₃P═CHCOOMe (1.43 g, 4.28 mmol) in 30 mL of THF was refluxed overnight. The reaction was cooled to room temperature, diluted with ethyl acetate, washed with brine, and dried over anhydrous magnesium sulfate. After concentration, the crude product was purified by flash chromatography (hexane:CH₂Cl₂:diethyl ether=40:60:3). 1.63 g (84%) of product was obtained as a white solid, ¹H NMR (CDCl₃, 500 MHz) δ 7.78 (s, 1H), 7.70 (d, J=15.69 Hz, 1H), 7.33 (m, 9H), 7.20 (m, 3H), 7.13 (m, 8H), 6.13 (d, J =15.69 Hz, 1H); ¹³C NMR (CDCl₃, 125 MHz) δ 167.18, 160.16, 148.51, 141.39, 139.18, 137.44, 136.90, 131.64, 130.41, 128.91, 128.68, 128.53, 127.93, 126.84, 125.65, 117.01, 83.45, 51.82, 35.56.

(Z) and (E)-methyl 3-(5-benzyl-4-(1H-tetrazol-5-yl)thiophen-2-yl)acrylate (DNM0194) A suspension of (E)-methyl 3-(5-benzyl-4-(2-trityl-2H-tetrazol-5-yl)thiophen-2-yl)acrylate (11.36 g, 20.0 mmol) in 100 mL of methanol was refluxed overnight. After being cooled to room temperature, the reaction mixture was concentrated to small amount of methanol left. Ether was added. After being stirred for 1 h, the solid was collected by suction filtration and dried under vacuum to give 5.87 g (90%) of product obtained as a white solid, ¹H NMR (DMSO, 500 MHz) δ 7.90 (s, 1H), 7.80 (d, J=15.76 Hz, 1H), 7.35 (m, 4H), 7.28 (m, 1H), 6.26 (d, J=15.77 Hz, 1H), 4.66 (s, 2H), 3.74 (s, 3H); ¹³C NMR (DMSO, 125 MHz) δ 166.10, 149.88, 139.10, 137.18, 136.30, 131.39, 128.64, 128.62, 126.81, 116.95, 51.53, 34.53.

### Example 2

### Synthesis of DNM0195

Following the procedure of Example 1: (Z) and (E)-3-(5-benzyl-4-(1H-tetrazol-5-yl)thiophen-2-yl)acrylic acid (DNM0195) To a solution of (Z) and (E)-methyl 3-(5-benzyl-4-(1H-tetrazol-5-yl)thiophen-2-yl)acrylate (5.54 g, 17.0 mmol) in 50 mL of methanol was added a solution of LiOH (2.04 g, 85.0 mmol) in 20 mL of H₂O. The reaction was stirred at room temperature, and the progress of the reaction was monitored by TLC until the reaction was complete. After removing most of methanol, the solution was acidified with 4N HCl. The resulting white solid was collected, and washed with water and ether, and dried under vacuum to give 4.77 g (90%) of product as a white solid, ¹H NMR (500 MHz, DMSO) δ 7.90 (s, 1H), 7.77 (d, J=15.73 Hz, 1H), 7.39 (m, 4H), 7.33 (m, 1H), 6.20 (d, J=15.73 Hz, 1H), 4.71 (s, 2H); ¹³C NMR (CDCl₃, 125 MHz) δ 166.90, 149.50, 139.15, 137.44, 135.70, 130.85, 128.65, 128.62, 126.80, 118.53, 34.53.

### Example 3

### Synthesis of DNM0226/DNM0227

Following the procedure of Example 1: (E)-methyl 3-(5-benzyl-4-(1-methyl-1H-tetrazol-5-yl)thiophen-2-yl)acrylate (DNM0227) and (E)-methyl 3-(5-benzyl-4-(2-methyl-2H-tetrazol-5-yl)thiophen-2-yl)acrylate (DNM0226) To a stirred suspension of (E)-methyl 3-(5-benzyl-4-(1H-tetrazol-5-yl)thiophen-2-yl)acrylate (326 mg, 1.0 mmol) and potassium carbonate (166 mg, 1.2 mmol) in 5 mL of dry DMF was added methyl iodide (125 μL, 2.0 mmol). The reaction was stirred at room temperature until complete. The reaction was diluted with ethyl acetate, and filtered. The filtrate was washed with H₂O and brine, dried over anhydrous sodium sulfate, and concentrated. The flash chromatography purification (CH2Cl2:EtOAc=20:1, then 20:3) afforded 64 mg (18.8%) of (E)-methyl 3-(5-benzyl-4-(1-methyl-1H-tetrazol-5-yl)thiophen-2-yl)acrylate as mono product and 260 mg (76.5%) of (E)-methyl 3-(5-benzyl-4-(2-methyl-2H-tetrazol-5-yl)thiophen-2-yl)acrylate as a white solid, ¹H NMR (DMSO, 500 MHz) δ 7.96 (s, 1H), 7.82 (d, J=15.68 Hz, 1H), 7.32 (m, 4H), 7.25 (m, 1H), 6.21 (d, J=15.69 Hz, 1H), 4.63 (s, 2H), 4.45 (s, 3H), 3.71 (s, 3H); ¹³C NMR (DMSO, 125 MHz) δ 166.17, 160.48, 148.30, 139.23, 137.08, 136.68, 131.81, 128.62, 128.60, 126.75, 125.07, 116.56, 51.46, 39.61, 34.55.

### Example 4

### Synthesis of DNM0197

Following the procedure of Example 1: (E)-3-(5-benzyl-4-(2-methyl-2H-tetrazol-5-yl)thiophen-2-yl)acrylic acid (DNM0197) (E)-methyl 3-(5-benzyl-4-(2-methyl-2H-tetrazol-5-yl)thiophen-2-yl)acrylate (150 mg, 0.44 mmol) was dissolved in 3 mL of methanol and 3 mL of THF. A solution of lithium hydroxide (34 mg, 1.42 mmol) in 2 mL of H₂O was added. The reaction was stirred overnight at room temperature. The solvent was removed, and the residue was redissolved in 5 mL of H₂O. After being acidified with 1 N HCl, the white precipitate formed was collected by suction filtration and dried under vacuum. 136 mg (95%) of product was obtained as a white solid, ¹H NMR (DMSO, 500 MHz) δ 7.98 (s, 1H), 7.81 (d, J=15.75 Hz, 1H), 7.39 (m, 4H), 7.32 (m, 1H), 6.18 (d, J=15.76 Hz, 1H), 4.70 (s, 2H), 3.52 (s, 3H); ¹³C NMR (CDCl₃, 125 MHz) δ 166.98, 160.52, 147.84, 139.28, 137.34, 136.11, 131.32, 128.62, 128.59, 126.74, 125.00, 118.11, 39.61, 34.54.

### Example 5

### Synthesis of DNM0196

Following the procedure of Example 1: (E)-3-(5-benzyl-4-(1-methyl-1H-tetrazol-5-yl)thiophen-2-yl)acrylic acid (DNM0196) (E)-methyl 3-(5-benzyl-4-(1-methyl-1H-tetrazol-5-yl)thiophen-2-yl)acrylate (40 mg, 0.12 mmol) was dissolved in 2 mL of methanol and 2 mL of THF. A solution of lithium hydroxide (15 mg, 0.63 mmol) in 1 mL of H₂O was added. The reaction was stirred overnight at room temperature. The solvent was removed, and the residue was re-dissolved in 5 mL of H₂O. After being acidified with 1 N HCl, the white precipitate formed was collected by suction filtration and dried under vacuum. 35 mg (91%) of product was obtained as a white solid.

### Example 6

### Synthesis of DNM0224

Methods A and B or method C were Used.

5-(2-Iodothiophen-3-yl)-2-trityl-2H-tetrazole A solution of 5-(Thiophen-3-yl)-2-trityl-2H-tetrazole (7.88 g, 20 mmol) in 125 mL of THF was cooled in a dry ice/acetone bath. To the solution was added a solution of n-BuLi (9.0 mL, 2.5 M, 22.5 mmol) dropwise. After addition was complete, the solution was stirred for 1 h at −78° C. Iodine (6.0 g, 23.6 mmol) was added. The reaction was stirred for 1 h at −78° C., quenched with saturated NH₄Cl, extracted with EtOAc. The organic phase was dried over anhydrous MgSO₄ and concentrated. The residue was purified by flash chromatography (hexane:CH₂Cl₂:diethyl ether=200:100:3). 9.50 g (91%) of product was obtained as a light yellow solid, ¹³C NMR (CDCl₃, 125 MHz) δ 159.83, 141.30, 133.21, 132.00, 130.40, 128.86, 128.34, 127.76, 83.39, 75.41.

5-(2-(4-Fluorophenyl)thiophen-3-yl)-2-trityl-2H-tetrazole To a solution of zinc chloride (6.75 mL, 1 M ether solution, 6.75 mmol) in 15 mL of THF was added a solution of 4-fluorophenylmagnesium bromide (2.25 mL, 2 M THF solution, 4.5 mmol) slowly. The resulting white suspension was stirred 10 min at room temperature, then transferred to a solution of 5-(2-iodothiophen-3-yl)-2-trityl-2H-tetrazole (1.775 g, 3.41 mmol) and Pd(PPh₃)₄ (158 mg, 0.14 mmol) in 10 mL of THF. The reaction progress was monitored using TLC. After complete, the reaction was quenched with saturated NH₄Cl, extracted with EtOAc. The organic phase was dried over anhydrous MgSO₄ and concentrated. The residue was purified by flash chromatography (hexane:CH₂Cl₂=5:3). 1.25 g (75%) of product was obtained as a white solid.

(E)-3-(5-(4-fluorophenyl)-4-(1H-tetrazol-5-yl)thiophen-2-yl)acrylic acid (DNM0224) Prepared in the same way as (E)-3-(5-benzyl-4-(1H-tetrazol-5-yl)thiophen-2-yl)acrylic acid in example 1. ¹H NMR (500 MHz, DMSO) δ 7.36 (s, 1H), 7.28 (d, J=14.15 Hz, 1H), 7.01 (m, 2H), 6.84 (m, 2H), 5.86 (d, J=14.15 Hz, 1H); ¹³C NMR (125 MHz, DMSO) δ 116.90, 163.57, 161.61, 145.00, 138.64, 135.49, 132.83, 131.28, 131.21, 128.12, 128.08, 119.34, 115.89, 115.72.

### Example 7

### Synthesis of DNM0225

Methods A and B or method C were Used.

5-(2-(4-Biphenylyl)thiophen-3-yl)-2-trityl-2H-tetrazole A solution of 4-biphenylyl boric acid (2.38 g, 12.0 mmol) and Pd(PPh₃)₄ (462 mg, 0.40 mmol) in 40 mL of 1,4-dioxane was stirred for 30 min under argon. 0.42 mL of H₂O was added. The solution was stirred for another 30 min. Potassium carbonate (3.45 g, 25.0 mmol) and 5-(2-iodothiophen-3-yl)-2-trityl-2H-tetrazole (5.20 g, 10.0 mmol) were sequentially added. The reaction was refluxed for 3 h. TLC showed that all of starting material was consumed. The reaction was cooled to room temperature, quenched with saturated brine, extracted with EtOAc. The organic phase was dried over anhydrous MgSO₄ and concentrated. The residue was purified by flash chromatography (hexane:ethyl acetate=10:1). 4.64 g (85%) of product was obtained as a white solid, ¹H NMR (CDCl₃, 500 MHz) δ 7.67 (d, J=5.33 Hz, 1H), 7.56 (m, 2H), 7.39-7.49 (m, 6H), 7.37 (m, 2H), 7.20-7.32 (m, 9H), 7.02 (m, 6H); ¹³C NMR (CDCl₃, 125 MHz) δ 160.73, 144.10, 141.35, 140.78, 140.62, 132.36, 130.32, 130.29, 129.34, 128.83, 128.27, 127.69, 127.46, 127.05, 126.72, 125.10, 124.29, 83.08.

(E)-3-(5-(4-biphenylyl)-4-(1H-tetrazol-5-yl)thiophen-2-yl)acrylic acid (DNM0225) Prepared in the same way as (E)-3-(5-benzyl-4-(1H-tetrazol-5-yl)thiophen-2-yl)acrylic acid in example 1. ¹H NMR (500 MHz, DMSO) δ 7.88 (s, 1H), 7.81 (d, J=15.71 Hz, 1H), 7.74 (m, 4H), 7.50 (m, 4H), 7.41 (m, 1H), 6.34 (d, J=15.77 Hz, 1H); ¹³C NMR (125 MHz, DMSO) δ 166.92, 145.87, 140.82, 139.01, 138.64, 135.53, 133.22, 130.68, 129.28, 129.00, 127.91, 127.00, 126.64, 119.31.

### Example 8

### Synthesis of DNM0234

Methods A and B or method C were Used.

5-(2-(Tributylstannyl)thiophen-3-yl)-2-trityl-2H-tetrazole A solution of 5-(Thiophen-3-yl)-2-trityl-2H-tetrazole (3.94 g, 10 mmol) in 40 mL of THF was cooled in a dry ice/acetone bath. To the solution was added dropwise a solution of n-BuLi (5.0 mL, 2.5 M, 12.5 mmol). After addition was complete, the solution was stirred for 2 h at −78° C. Trbutyltin chloride (3.4 mL, 12.5 mmol) was added. The reaction was stirred for 1 h at −78° C., quenched with brine, extracted with EtOAc. The organic phase was dried over anhydrous MgSO₄ and concentrated. The residue was purified by flash chromatography (hexane:diethyl ether=20:1). 6.24 g (91%) of product was obtained as a colorless oil, ¹H NMR (CDCl₃, 500 MHz) δ 7.83 (d, J=4.78 Hz, 1H), 7.63 (d, J=4.81 Hz, 1H), 7.33 (m, 9H), 7.15 (m, 6H), 1.42 (m, 6H), 1.23 (m, 6H), 1.03 (t, J=8.19 Hz, 6H), 0.90 (t, J=7.32 Hz, 9H); ¹³C NMR (CDCl₃, 125 MHz) δ 162.65, 141.66, 140.42, 135.67, 131.49, 130.41, 129.09, 128.38, 127.93, 82.97, 29.04, 27.30, 13.81, 11.69.

5-(2-(Naphthalen-1-yl)thiophen-3-yl)-2-trityl-2H-tetrazole A solution of 5-(2-(tributylstannyl)thiophen-3-yl)-2-trityl-2H-tetrazole (2.73 g, 4.0 mmol), 2-iodonaphthalene (0.88 mL, 6.0 mmol), Pd(PPh₃)₄ (139 mg, 0.12 mmol) and CuI (45.6 mg, 0.24 mmol) in 12 mL of DMF was heated to 60° C. under argon overnight. The reaction was cooled to room temperature, quenched with saturated H₂O, and extracted with EtOAc. The organic phase was dried over anhydrous MgSO₄ and concentrated. The residue was purified by flash chromatography (hexane:CH₂Cl₂:ether=50:25:3). 1.73 g (83%) of product was obtained as a white solid.

(E)-methyl 3-(5-(naphthalen-1-yl)-4-(1H-tetrazol-5-yl)thiophen-2-yl)acrylate (DNM0234) Prepared in the same way as (E)-methyl 3-(5-benzyl-4-(1H-tetrazol-5-yl)thiophen-2-yl)acrylate in example 1. ¹H NMR (500 MHz, DMSO) δ 7.58 (s, 1H), 7.54 (m, 1H), 7.49 (d, J=7.78 Hz, 1H), 7.43 (d, J=14.22 Hz, 1H), 7.12 (m, 2H), 7.05 (dd, J₁=5.63 Hz, J₂=8.44 Hz, 2H), 6.95 (m, 1H), 6.09 (d, J=14.22 Hz, 1H), 3.66 (s, 3H); ¹³C NMR (125 MHz, DMSO) δ 166.11, 143.60, 139.29, 136.19, 133.12, 131.91, 131.15, 129.79, 129.20, 128.93, 128.39, 126.84, 126.19, 125.29, 124.56, 117.87, 51.61.

### Example 9

### Synthesis of DNM0235

Following the procedure of Example 8: (E)-3-(5-(naphthalen-1-yl)-4-(1H-tetrazol-5-yl)thiophen-2-yl)acrylic acid (DNM0235) Prepared in the same way as (E)-3-(5-benzyl-4-(1H-tetrazol-5-yl)thiophen-2-yl)acrylic acid in example 2. ¹H NMR (500 MHz, DMSO) δ 8.10 (m, 2H), 8.04 (m, 1H), 7.90 (d, J=14.76 Hz, 1H), 7.63 (m, 2H), 7.56 (m, 2H), 7.44 (m, 1H), 6.38 (d, J=15.18 Hz, 1H); ¹³C NMR (125 MHz, DMSO) δ 167.47, 143.71, 140.09, 136.12, 133.65, 131.96, 131.71, 130.28, 129.78, 129.46, 128.92, 127.36, 126.71, 125.82, 125.12, 119.97.

### Example 10

### Synthesis of DNM0236

Following the procedure of Example 8: (E)-methyl 3-(4-(2-methyl-2H-tetrazol-5-yl)-5-(naphthalen-1-yl)thiophen-2-yl)acrylate (DNM0236) Prepared analogous to (E)-methyl 3-(5-benzyl-4-(2-methyl-2H-tetrazol-5-yl)thiophen-2-yl)acrylate (DNM0226) in Example 3. ¹H NMR (500 MHz, CDCl₃) δ 7.96-7.90 (m, 2H), 7.88 (d, J=8.16 Hz, 1H), 7.84 (d, J=15.74 Hz, 1H), 7.62 (d, J=8.46 Hz, 1H), 7.57 (dd, J₁=7.24 Hz, J₂=1.24 Hz, 1H), 7.54-7.49 (m, 1H), 7.48-7.42 (m, 1H), 7.37-7.30 (m, 1H), 6.30 (d, J=15.72 Hz, 1H), 4.04 (s, 3H), 3.82 (s, 3H); ¹³C NMR (125 MHz, CDCl₃) δ 167.04, 160.97, 143.91, 139.43, 136.61, 133.52, 132.14, 131.36, 130.38, 129.69, 128.85, 128.33, 127.46, 126.55, 126.03, 125.48, 125.06, 117.71, 51.78, 39.18.

### Example 11

### Synthesis of DNM0229

### Method D was Used.

2-Bromo-5-tributylstannylthiophene A solution of 2-bromothiophene (5 mL, 51.6 mmol) in 125 mL of THF was cooled to −78° C. LDA (28.5 mL, 2M in THF, 57 mmol) was added dropwise. After addition was complete, the reaction mixture was stirred for 1 h at −78° C. Tributylstannyl chloride (15.0 mL, 55.3 mmol) was added. After being stirred for 1 h at −78° C., quenched with saturated bicarbonate, and extracted with ethyl acetate. The acetate layer was dried over anhydrous MgSO₄ and concentrated. The residue was purified by flash chromatography (hexane). 16.6 g (71%) of product was obtained as a colorless oil.

2-Bromo-5-(4-fluorophenyl)thiophene A solution of 2-bromo-5-tributylstannylthiophene (2.26 g, 5 mmol), 1-fluoro-4-iodobenzene (0.9 mL, 7.80 mmol), Pd(PPh₃)₄ (175 mg, 0.15 mmol) and CuI (57 mg, 0.30 mmol) in 15 mL of DMF was heated to 60° C. under argon overnight. After being cooled to room temperature, the reaction was quenched with H₂O, and extracted with ethyl acetate. The acetate layer was dried over anhydrous magnesium sulfate. After concentration, the residue was purified by flash chromatography (hexane) to afford 1.05 g (82%) of product as white solid, ¹H NMR (CDCl₃, 500 MHz) δ 7.47 (m, 2H), 7.06 (m, 2H), 7.01 (d, J=3.84 Hz, 1H), 6.96 (d, J=3.84 Hz, 1H); ¹³C NMR (CDCl₃, 125 MHz) δ 163.52, 161.54, 144.78, 130.89, 130.00, 129.97, 127.43, 127.37, 123.29, 138.28, 116.12, 115.95, 111.36.

3-Bromo-5-(4-fluorophenyl)thiophene-2-carbaldehyde A solution of 2-bromo-5-(4-fluorophenyl)thiophene (1.00 g, 3.89 mmol) was cooled to −78° C. LDA (2.35 mL, 2M in THF, 4.70 mmol) was added dropwise. After addition was complete, the reaction mixture was stirred for 45 min at −78° C. DMF (0.9 mL, 11.6 mmol) was added. The reaction was warmed to room temperature, stirred at room temperature until complete, quenched with saturated NH₄Cl, and extracted with ethyl acetate. The acetate layer was dried over anhydrous MgSO₄ and concentrated. The residue was purified by flash chromatography (hexane:ether=10:1) to give 0.85 g (77%) of product as a white solid.

5-(4-Fluorophenyl)-2-formylthiophene-3-carbonitrile A mixture of 3-bromo-5-(4-fluorophenyl)thiophene-2-carbaldehyde (0.74 g, 2.60 mmol) and CuCN (0.70 g, 7.80 mmol) in 7.5 mL of DMF was heated to 150° C. TLC after 4 h showed that all of the starting material was consumed. The reaction was cooled to room temperature, quenched with 1 N HCl, stirred for 30 min, and extracted with ethyl acetate. The acetate layer was dried over anhydrous MgSO₄ and concentrated. The residue was purified by flash chromatography (hexane:dichloromethane=1:4) to give 523 mg (87%) of product as a white solid.

5-(4-Fluorophenyl)-3-(1H-tetrazol-5-yl)thiophene-2-carbaldehyde A mixture of 5-(4-fluorophenyl)-2-formylthiophene-3-carbonitrile (510 mg, 2.2 mmol), sodium azide (293 mg, 4.5 mmol) and zinc bromide (1.01 g, 4.5 mmol) in 12.5 mL was heated to 100° C. TLC after 6 h showed that all of the starting material was consumed. The reaction was cooled to room temperature, quenched 1 N HCl, stirred for 30 min, and extracted with ethyl acetate. The acetate layer was dried over anhydrous MgSO₄ and concentrated. The residue was purified by flash chromatography (hexane:ethyl acetate:acetic acid=50:50:2) to give 512 mg (87%) of product as a white solid.

(E)-methyl 3-(5-(4-fluorophenyl)-3-(1H-tetrazol-5-yl)thiophen-2-yl)acrylate (DNM0229) A mixture of 5-(4-fluorophenyl)-3-(1H-tetrazol-5-yl)thiophene-2-carbaldehyde (610 mg, 2.23 mmol) and Ph₃P═CHCOOMe (895 mg, 2.99 mmol) in 15 mL of THF was refluxed. TLC after 5 h showed that all of the starting material was consumed. The reaction was cooled to room temperature, quenched 1 N HCl, stirred for 10 min. The resulting yellow solid was collected and purified by flash chromatography (hexane:THF:acetic acid=15:25:1) to give 600 mg (69%) of yellow solid as acetic acid adduct, ¹H NMR (500 MHz, DMSO) δ 8.68 (d, J=15.82 Hz, 1H), 8.01 (s, 1H), 7.82 (m, 2H), 7.40 (m, 2H), 6.48 (d, J=15.72 Hz, 1H), 3.79 (s, 3H), 1.95 (s, 3H); ¹³C NMR (125 MHz, DMSO) δ 171.91, 165.96, 163.49, 161.52, 144.58, 137.24, 135.30, 128.57, 128.01, 127.94, 124.68, 118.89, 116.55, 116.38, 51.71, 20.98.

### Example 12

### Synthesis of DNM0230

Following the procedure of Example 11: (E)-3-(5-(4-fluorophenyl)-3-(1H-tetrazol-5-yl)thiophen-2-yl)acrylic acid (DNM0230) (E)-methyl 3-(5-(4-fluorophenyl)-3-(1H-tetrazol-5-yl)thiophen-2-yl)acrylate (120 mg, 0.30 mmol) was dissolved in 5 mL of methanol. A solution of lithium hydroxide (36 mg, 1.50 mmol) in 2 mL of H₂O was added. The reaction was stirred overnight at room temperature. The solvent was removed, and the residue was redissolved in 5 mL of H₂O. After being acidified with 1 N HCl, the white precipitate formed was collected by suction filtration and dried under vacuum. 90 mg (95%) of product was obtained as a yellow solid, ¹H NMR (DMSO, 500 MHz) 8.61 (d, J=15.77 Hz, 1H), 7.97 (s, 1H), 7.80 (m, 2H), 7.37 (m, 2H), 6.35 (d, J=15.77 Hz, 1H); ¹³C NMR (125 MHz, DMSO) δ 166.87, 163.44, 161.47, 144.15, 137.39, 134.98, 128.67, 127.98, 127.91, 127.65, 124.68, 120.32, 116.53, 116.35.

### Example 13

### Synthesis of DNM0231

Following the procedure of Example 11: (E)-methyl 3-(5-(4-fluorophenyl)-3-(2-methyl-2H-tetrazol-5-yl)thiophen-2-yl)acrylate (DNM0231) To a stirred suspension of (E)-3-(5-(4-fluorophenyl)-3-(1H-tetrazol-5-yl)thiophen-2-yl)acrylic acid (700 mg, 1.79 mmol) and potassium carbonate (690 mg, 5.0 mmol) in 7.5 mL of dry DMF was added methyl iodide (0.3 mL, 4.82 mmol). The reaction was stirred at room temperature until complete. The reaction was diluted with ethyl acetate, and filtered. The filtrate was washed with H₂O and brine, dried over anhydrous sodium sulfate, and concentrated. The flash chromatography purification (hexane: CH2Cl2:EtOAc=20:10:3, then 20:10:6) afforded 60 mg of (E)-methyl 3-(5-(4-fluorophenyl)-3-(1-methyl-1H-tetrazol-5-yl)thiophen-2-yl)acrylate as mono product and 380 mg of (E)-methyl 3-(5-(4-fluorophenyl)-3-(2-methyl-2H-tetrazol-5-yl)thiophen-2-yl)acrylate as a yellow solid, ¹H NMR (DMSO, 500 MHz) δ 8.65 (d, J=15.77 Hz, 1H), 8.00 (s, 1H), 7.85 (m, 2H), 7.33 (m, 2H), 6.41 (d, J=15.77 Hz, 1H), 4.49 (s, 1H), 3.76 (s, 3H); ¹³C NMR (125 MHz, DMSO) δ 166.02, 163.43, 161.47, 160.18, 144.52, 136.07, 135.44, 130.22, 128.61, 128.13, 128.06, 124.54, 118.50, 116.37, 116.19, 51.67, 39.78.

### Example 14

### Synthesis of DNM0232

Following the procedure of Example 11: (E)-3-(5-(4-fluorophenyl)-3-(2-methyl-2H-tetrazol-5-yl)thiophen-2-yl)acrylic acid (DNM0232) Prepared by cleavage of methyl ester of (E)-methyl 3-(5-(4-fluorophenyl)-3-(2-methyl-2H-tetrazol-5-yl)thiophen-2-yl)acrylate as a yellow solid, ¹H NMR (DMSO, 500 MHz) δ 12.58 (s, 1H), 8.59 (d, J=15.73 Hz, 1H), 7.97 (s, 1H), 7.83 (m, 2H), 7.32 (m, 2H), 6.31 (d, J=15.73 Hz, 1H), 4.48 (s, 3H); ¹³C NMR (125 MHz, DMSO) δ 166.87, 163.38, 161.42, 160.23, 144.11, 136.43, 134.96, 129.78, 128.69, 128.07, 128.00, 124.47, 120.08, 116.33, 116.16, 39.77.

### Example 15

### Synthesis of DNM0233

Following the procedure of Example 11: (E)-3-(5-(4-fluorophenyl)-3-(1-methyl-1H-tetrazol-5-yl)thiophen-2-yl)acrylic acid (DNM0233) Prepared by cleavage of methyl ester of (E)-methyl 3-(5-(4-fluorophenyl)-3-(1-methyl-1H-tetrazol-5-yl)thiophen-2-yl)acrylate as a yellow solid, ¹H NMR (DMSO, 500 MHz) δ 8.00 (s, 1H), 7.91 (d, J=15.75 Hz, 1H), 7.87 (m, 2H), 7.38 (m, 2H), 6.35 (d, J=15.74 Hz, 1H), 3.79 (s, 3H), 1.95 (s, 3H); ¹³C NMR (125 MHz, DMSO) δ 166.74, 163.50, 161.53, 149.45, 144.41, 139.35, 134.32, 128.66, 128.63, 128.22, 128.16, 125.92, 125.00, 120.41, 116.44, 116.26, 35.01.

### Example 16

### Synthesis of DNM0240

### Method D was Used.

2-(Biphenyl-4-yl)-5-bromothiophene A solution of (4-biphenyl)boric acid (1.90 g, 9.60 mmol) and Pd(Ph₃)₄ (0.37 g, 0.32 mmol) in 20 mL of DME was degassed and stirred for 0.5 h. To the resultant solution, 0.35 mL water was added. After stirring for 0.5 h, sodium carbonate (2.12 g, 20.0 mmol) and 2,5-dibromothiophene (0.90 mL, 8.0 mmol) was sequentially. The reaction mixture was refluxed for 24 h. After cooled to rt, brine was added to the reaction mixture. The resultant suspension was extracted with dichloromethane. The organic phase was dried over anhydrous sodium sulfate, concentrated, and the residue was purified by flash chromatography (hexane). 1.30 g (51.6%) product was obtained as a light yellow solid, ¹H NMR (CDCl₃, 500 MHz) δ 7.63-7.55 (m, 6H), 7.48-7.41 (m, 2H), 7.39-7.33 (m, 1H), 7.09 (d, J=3.84 Hz, 1H), 7.04 (d, J=3.84 Hz, 1H); ¹³C NMR (CDCl₃, 125 MHz) δ 145.55, 140.72, 140.37, 132.65, 130.96, 128.89, 127.69, 127.57, 127.40, 126.94, 125.99, 123.28, 111.49, 68.20.

(E)-methyl 3-(5-(4-biphenyl)-3-(1H-tetrazol-5-yl)thiophen-2-yl)acrylate (DNM0240) Prepared in the same way as (E)-methyl 3-(5-(4-fluorophenyl)-3-(1H-tetrazol-5-yl)thiophen-2-yl)acrylate (DNM0229) in example 11. ¹H NMR (DMSO, 500 MHz) δ 8.67 (d, J=15.81 Hz, 1H), 8.06 (s, 1H), 7.82 (m, 4H), 7.74 (d, J=7.7 Hz, 2H), 7.50 (m, 2H), 7.40 (m, 1H), 6.45 (d, J=15.81 Hz, 1H), 3.75 (s, 3H); ¹³C NMR (DMSO, 125 MHz) δ 165.99, 145.33, 140.74, 138.95, 137.19, 135.36, 130.91, 129.00, 127.90, 127.56, 126.54, 126.21, 124.56, 118.84, 51.70.

### Example 17

### Synthesis of DNM0245

Following the procedure of example 16: (E)-3-(5-(biphenyl-4-yl)-3-(1H-tetrazol-5-yl)thiophen-2-yl)acrylic acid (DNM0245) Prepared by cleavage of methyl ester of (E)-methyl 3-(5-(4-biphenyl)-3-(1-methyl-1H-tetrazol-5-yl)thiophen-2-yl)acrylate (DNM0240) as a yellow solid, ¹H NMR (DMSO, 500 MHz) δ 8.63 (d, J=15.76 Hz, 1H), 8.08 (s, 1H), 7.84 (s, 4H), 7.78-7.73 (m, 2H), 7.54-7.48 (m, 2H), 7.44-7.39 (m, 1H), 6.39 (d, J=15.76 Hz, 1H); ¹³C NMR (DMSO, 125 MHz) δ 166.77, 144.89, 140.60, 138.88, 137.53, 134.78, 130.88, 128.91, 127.80, 127.47, 126.45, 126.11, 124.44, 120.38.

### Example 18

### Synthesis of DNM0243

### Method D was Used.

2-Benzyl-5-bromothiophene A solution 2-bromothiophene (4.8 mL, 49.6 mmol) and TMEDA (9.0 mL, 60.0 mmol) in 150 mL of THF was cooled to −78° C. To the resultant solution, LDA (30 mL, 2M, 60.0 mmol) was dropwise added at −78° C. After complete addition, the reaction mixture was stirred for 1 h at −78° C., then benzyl bromide (9.0 mL, 75.7 mmol) was added. The reaction mixture was warmed to rt naturally, and stirred overnight at rt. The reaction was worked using typical procedure, and the crude product was purified by flash chromatography (hexane). 7.71 g product was obtained as a clear oil.

(E)-methyl 3-(5-benzyl-3-(1H-tetrazol-5-yl)thiophen-2-yl)acrylate (DNM0243) Prepared in the same way as (E)-methyl 3-(5-(4-fluorophenyl)-3-(1H-tetrazol-5-yl)thiophen-2-yl)acrylate (DNM0229) in example 11. ¹H NMR (DMSO, 500 MHz) δ 8.63 (d, J=15.82 Hz, 1H), 7.42 (s, 1H), 7.40-7.32 (m, 4H), 7.31-7.25 (m, 1H), 6.31 (d, J=15.82 Hz, 1H), 4.25 (s, 2H), 3.73 (s, 3H); ¹³C NMR (DMSO, 125 MHz) δ 165.95, 148.27, 138.98, 136.88, 135.50, 128.59, 128.53, 126.70, 126.39, 118.10, 51.52, 35.21.

### Example 19

### Synthesis of DNM0246

### Method D was Used.

5-((5-benzyl-3-(1H-tetrazol-5-yl)thiophen-2-yl)methylene)thiazolidine-2,4-dione (DNM0246) A mixture of 5-benzyl-3-(1H-tetrazol-5-yl)thiophene-2-carbaldehyde (135 mg, 0.5 mmol), mmol), thiazolidine-2,4-dione (65 mg, 90%, 0.5 mmol), ammonium acetate (43 mg, 0.55 mmol), and acetic acid (0.5 mL) in 10 mL of benzene was heated under reflux with azeotropic removal of water using a Dean-Stark water collector. After 0.5 h, additional 5 mL of benzene was added to the reaction mixture, and the reaction continued for a further 2 h. After cooled to rt, the reaction mixture was concentrated. The residue was suspended in 1N HCl, and extracted with ethyl acetate. The organic phase was dried over anhydrous MgSO₄ and concentrated. The residue was purified by flash chromatography (hexane:ethyl acetate:acetic acid=100:60:4) and 117 mg (63%) of product was obtained as a yellow solid, ¹H NMR (DMSO, 500 MHz) δ 12.73 (broad, 1H), 8.94 (s, 1H), 7.56 (s, 1H), 7.45-7.22 (m, 5H), 4.34 (s, 2H); ¹³C NMR (DMSO, 125 MHz) δ 166.87, 166.66, 151.42, 138.94, 134.98, 128.66, 128.57, 126.78, 126.76, 123.43, 123.38, 35.23.

### Example 20

### Synthesis of DNM0264

### Method D was Used.

Diethyl 2-((5-benzyl-4-(1H-tetrazol-5-yl)thiophen-2-yl)methylene)malonate A mixture of 5-benzyl-3-(1H-tetrazol-5-yl)thiophene-2-carbaldehyde (1.88 g, 6.95 mmol), diethyl malonate (1.30 mL, 8.34 mmol), piperidine (0.34 mL, 3.48 mmol) and acetic acid (0.08 mL, 1.44 mmol) in 30 mL of benzene was heated under reflux with azeotropic removal of water using a Dean-Stark water collector overnight. After cooled to rt, the reaction mixture was concentrated, and the residue was purified by flash chromatography, and 1.92 g (67%) of product was obtained a white solid.

2-((5-benzyl-4-(1H-tetrazol-5-yl)thiophen-2-yl)methylene)malonic acid (DNM0264) A mixture of diethyl 2-((5-benzyl-4-(1H-tetrazol-5-yl)thiophen-2-yl)methylene)malonate (412 mg, 1.0 mmol) and LiOH.H₂O (210 mg, 5.0 mmol) in 10 mL of water-ethanol (1:1) was heated to 70° C., and the reaction was monitored by TLC. After the reaction was complete, the reaction mixture was concentrated. The residue was re-dissolved 15 mL of water, and filtered. The filtrate was acidified with 1N HCl, the resultant precipitate was collected by suction filtration, washed with water and diethyl ether, and dried under vacuum to give 320 mg (90%) of product as a white solid, ¹H NMR (DMSO, 500 MHz) δ 7.93 (s, 1H), 7.74 (s, 1H), 7.50-7.05 (m, 5H), 4.65 (s, 2H); ¹³C NMR (DMSO, 125 MHz) δ 167.35, 165.59, 152.40, 139.04, 134.80, 134.50, 132.26, 128.57, 126.74, 124.38, 121.80, 34.43.

### Example 21

### Synthesis of DNM0252

Methods A and B were Used.

5-(2-Benzyl-5-iodothiophen-3-yl)-2-trityl-2H-tetrazole A solution of 5-(2-benzylthiophen-3-yl)-2-trityl-2H-tetrazole (4.085 g, 8.44 mmol) and TMEDA (1.5 mL, 9.86 mmol) in 50 mL of THF was cooled in a dry ice/acetone bath. A solution of t-BuLi (5.80 mL, 1.7 M, 9.86 mmol) was added dropwise. After addition was complete, the solution was stirred for 1 h at −78° C. To the solution was added I₂ (2.54 g, 10.0 mmol). The reaction was stirred for 1 h at −78° C., and then quenched with saturated NH₄Cl. The reaction mixture was extracted with EtOAc. The organic phase was dried over anhydrous MgSO₄ and concentrated. The residue was purified by flash chromatography (hexane:dichloromethane:ether=200:100:5), and 6.0 g (90%) of product was obtained as a white solid.

(E)-methyl 3-(5-benzyl-4-(2-(3-methoxy-3-oxopropyl)-2H-tetrazol-5-yl)thiophen-2-yl)acrylate A mixture of 5-(2-Benzyl-5-iodothiophen-3-yl)-2-trityl-2H-tetrazole (1.88 g, 3.08 mmol), methyl acrylate (1.80 mL, 20.0 mmol), palladium acetate (27 mg, 0.12 mmol), dppp (103 mg, 0.25 mmol) and triethylamine (1.4 mL, 10.0 mmol) in 15 mL of DMF was heated to 100° C. for 24 h. After cooled to rt, the reaction was worked up using a typical way, and the crude product was purified by flash chromatography to afford the product as a light yellow solid, ¹H NMR (CDCl₃, 500 MHz) δ 7.75 (s, 1H), 7.70 (d, J=15.69 Hz, 1H), 7.35-7.20 (m, 5H), 6.13 (d, J=15.68 Hz, 1H), 4.96 (t, J=6.95 Hz, 2H), 4.61 (s, 2H), 3.77 (s, 3H), 3.72 (s, 3H), 3.12 (t, J=6.95 Hz, 2H); ¹³C NMR (CDCl₃, 125 MHz) δ 171.10, 167.69, 161.50, 148.99, 139.17, 137.49, 136.76, 131.33, 128.95, 128.70, 126.98, 125.17, 116.95, 52.32, 51.74, 48.52, 35.74, 33.24.

(E)-3-(5-benzyl-4-(2-(2-carboxyethyl)-2H-tetrazol-5-yl)thiophen-2-yl)acrylic acid (DNM0252) Prepared by cleavage of methyl ester of (E)-methyl 3-(5-benzyl-4-(2-(3-methoxy-3-oxopropyl)-2H-tetrazol-5-yl)thiophen-2-yl)acrylate as a white solid, ¹H NMR (DMSO, 500 MHz) δ 7.91 (s, 1H), 7.75 (d, J=15.75 Hz, 1H), 7.35-7.20 (m, 5H), 6.12 (d, J=15.75 Hz, 1H), 4.93 (t, J=6.48 Hz, 2H), 4.61 (s, 2H), 3.09 (t, J=6.48 Hz, 2H); ¹³C NMR (DMSO, 125 MHz) δ 171.34, 166.98, 160.31, 147.97, 139.28, 137.34, 136.10, 131.29, 128.67, 128.59, 126.74, 124.92, 118.15, 48.80, 34.56, 32.57.

### Example 22

### Synthesis of DNM0270

### Method D was Used.

5-(2-Bromothiophen-3-yl)-1H-tetrazole To a solution of 5-(thiophen-3-yl)-1H-tetrazole (865 mg, 5.7 mmol) in 15 mL of AcOH and DMF (2:1), NBS (1.51 g, 8.5 mmol) was added. After stirred overnight at rt, the reaction mixture was poured onto crushed ice. The resultant solid was colleted by suction filtration, washed with water, and dried under vacuum. ¹H NMR (DMSO, 500 MHz) δ 8.05 (d, J=5.40 Hz, 1H), 7.52 (d, J=5.40 Hz, 1H); ¹³C NMR (DMSO, 125 MHz) δ 161.99, 133.57, 132.76, 130.41, 128.47.

2-Benzhydryl-5-(2-bromothiophen-3-yl)-2H-tetrazole A suspension of 5-(2-bromothiophen-3-yl)-1H-tetrazole (1.16 g, 5.0 mmol) and Ph₂CHOH (0.92 g, 5.0 mmol) in 25 mL of toluene with existence of catalytic amount of TsOH was heated under reflux until a clear solution formed. The resultant solution was cooled to rt, and concentrated under vacuum. The residue was purified by flash chromatography to afford a white solid, ¹H NMR (CDCl₃, 500 MHz) δ 7.78 (d, J=5.35 Hz, 1H), 7.46 (d, J=5.35 Hz, 1H), 7.23-7.17 (m, 2H), 7.17-7.10 (m, 4H), 7.00 (s, 1H), 6.94 (d, J=7.54 Hz, 4H); ¹³C NMR (CDCl₃, 125 MHz) δ 161.44, 137.17, 133.51, 129.08, 128.63, 128.43, 128.04, 127.67, 127.11, 70.83.

2-Benzhydryl-5-(2-(3-bromobenzyl)thiophen-3-yl)-2H-tetrazole A solution of 2-benzhydryl-5-(2-bromothiophen-3-yl)-2H-tetrazole (2.48 g, 6.24 mmol) in 25 mL of THF was cooled in an ice-salt bath. To this cooled solution, isopropylmagnesium chloride solution (3.60 mL, 2M, 7.20 mmol) was dropwise added. After the complete addition, the reaction continued for 1 h at the same temperature, and then a THF solution of 3-bromobenzyl iodide (2.32 g, 7.8 mmol) was added followed by Li₂CuCl₄ solution (0.1 M, 0.5 mL). The reaction was warmed to rt, and then heated to 40° C. for 2 h. After cooled to rt, the reaction was quenched with saturated ammonium chloride, and qorked up with a typical procedure. The crude product was purified by flash chromatography (hexane:dichloromethane=2:3) to give 2.60 g (85%) product as viscous oil, ¹H NMR (CDCl₃, 500 MHz) δ 7.65 (d, J=5.35 Hz, 1H), 7.39-7.22 (m, 13H), 7.19 (d, J=5.35 Hz, 1H), 7.12-7.02 (m, 2H), 4.52 (s, 2H); ¹³C NMR (CDCl₃, 125 MHz) δ 161.97, 143.55, 142.30, 137.17, 131.71, 130.01, 129.61, 128.85, 128.73, 128.30, 128.04, 127.35, 124.95, 127.09, 122.52, 71.15, 34.49.

2-Benzhydryl-5-(2-(3-bromobenzyl)-5-iodothiophen-3-yl)-2H-tetrazole A suspension of 2-benzhydryl-5-(2-bromothiophen-3-yl)-2H-tetrazole (1.76 g, 3.61 mmol), iodine (1.02 g, 4.0 mmol) and silver sulfate (1.25 g, 4.0 mmol) was stirred overnight at rt. The reaction mixture was filtered, and the filtrate was concentrated. The residue was purified by flash chromatography (1% ethyl aceate in hexane) to give 1.80 g (81%) product.

(E)-methyl 3-(3-((3-(2-benzhydryl-2H-tetrazol-5-yl)-5-((E)-3-methoxy-3-oxoprop-1-enyl)thiophen-2-yl)methyl)phenyl)acrylate A solution of 2-Benzhydryl-5-(2-(3-bromobenzyl)-5-iodothiophen-3-yl)-2H-tetrazole (433 mg, 0.71 mmol), palladium acetate (13 mg, 0.058 mmol), DPPP (48 mg, 0.12 mmol) in 7.5 mL of DMF was degassed. To the resultant solution was added methyl acrylate (0.64 mL, 7.1 mmol) and triethylamine (0.5 mL, 3.55 mmol). The reaction was heated to 120° C. for 12 h. After cooled to rt, the reaction was worked up with a typical procedure, and the crude product was purified by flash chromatography to give 164 mg (40%) product as a white solid, ¹H NMR (CDCl₃, 500 MHz) δ 7.79 (s, 1H), 7.70 (d, J=15.70 Hz, 1H), 7.61 (d, J=16.15 Hz, 1H), 7.42-7.20 (m, 16H), 6.38 (d, J=16.02 Hz, 1H), 6.15 (d, J=15.70 Hz, 1H), 4.58 (s, 2H), 3.79 (s, 3H), 3.77 (s, 3H); ¹³C NMR (CDCl₃, 125 MHz) δ 167.32, 167.02, 161.35, 147.36, 144.63, 139.94, 137.63, 137.00, 136.61, 134.80, 131.42, 130.76, 129.19, 128.85, 128.80, 128.57, 128.26, 126.42, 125.72, 118.10, 117.23, 71.26, 51.74, 51.70, 35.31.

(E)-3-(3-((5-((E)-2-carboxyvinyl)-3-(1H-tetrazol-5-yl)thiophen-2-yl)methyl)phenyl)acrylic acid (DNM0270) Prepared by cleavage of the methyl ester and benzhydryl protecting group of (E)-methyl 3-(3-((3-(2-benzhydryl-2H-tetrazol-5-yl)-5-((E)-3-methoxy-3-oxoprop-1-enyl)thiophen-2-yl)methyl)phenyl)acrylate. ¹H NMR (DMSO, 500 MHz) δ 12.42 (broad, 2H), 7.84 (s, 1H), 7.76-7.45 (m, 4H), 7.45-7.25 (m, 2H), 6.48 (d, J=15.40 Hz, 1H), 6.15 (d, J=15.51 Hz, 1H), 4.66 (s, 2H); ¹³C NMR (DMSO, 125 MHz) δ 167.33, 166.84, 143.55, 139.80, 137.47, 135.61, 134.46, 130.80, 130.47, 129.18, 128.37, 126.70, 119.35, 118.53, 34.28.

### Example 23

### Testing Compounds for Inhibition of the LpxA Enzyme

The procedure as given above was followed, with the appropriate dilutions of test compound being 5 mM and 500 μM. Measurement of E. coli LpxA activity was recorded as a percentage of control (lower is better):

| Compound | | |
|---|---|---|
| designation | % LpxA activity (5 mM) | % LpxA activity (500 μM) |
| DNM0194 | 1.4 | 36.0 |
| DNM0195 | 3.1 | 7.6 |
| DNM0196 | 5.8 | 43.9 |
| DNM0197 | 20.2 | 62.4 |
| DNM0224 | 37.9 | 88.7 |
| DNM0225 | 7.3 | 84.9 |
| DNM0226 | 93.0 | 97.0 |
| DNM0227 | 86.6 | 92.1 |
| DNM0229 | 4.0 | 57.8 |
| DNM0230 | 37.9 | 88.7 |
| DNM0233 | 47.3 | 98.8 |
| DNM0234 | 1.1 | 92.1 |
| DNM0235 | 7.3 | 59.6 |
| DNM0238 | 3.0 | 75.9 |
| DNM0239 | 9.5 | 76.8 |
| DNM0240 | 6.6 | 90.7 |
| DNM0243 | 0.9 | 69.2 |
| DNM0245 | 4.4 | 78.5 |
| DNM0246 | 10.3 | 165.2 |
| DNM0252 | 5.1 | 62.9 |
| DNM0264 | 5.4 | 56.8 |
| DNM0270 | 3.0 | 89.7 |

### Example 24

### Detecting Antibiotic Minimum Inhibitory Concentrations

Procedure followed was as described above, with the appropriate range being 1 μM-2 mM and using the E. coli D22 mutant strain and the E. coli K12 wild type strain. Value recorded was the lowest concentration of compound needed to produce the bactericidal or bacteriostatic effect (lower is better):

| Compound designation | Strain tested | MIC |
|---|---|---|
| DNM0194 | D22 | 1-2 mM |
| DNM0195 | D22 | 1 mM |
| DNM0195 | K12 | 1-2 mM |

### Example 25

### Detecting Pro-Antibiotic Minimum Inhibitory Concentrations

Following Example 24, MICs of various antibiotic agents were determined against the E. coli K12 wild type strain, using the same procedure with the appropriate range for each antibiotic. For erythromycin, gentamicin, and kanamycin, this range was 0.24 μg/mL-500 μg/mL. Parallel to this effort, solutions were prepared which were identical. To each of these were added 0.5 μL of DNM0195 stock solution and mixed to produce a final concentration of 500 μM for determination of pro-antibiotic activity. Compounds were determined to be pro-antibiotic if the mixture produced a lower MIC than that of the antibiotic agent alone. Results for these experiments follow (lower is better):

| Compound/mixture designation | MIC |
|---|---|
| Erythromycin | 62.5 μg/mL |
| Erythromycin + 500 μM DNM0195 | 0.48 μg/mL |
| Kanamycin | 3.125 μg/mL |
| Kanamycin + 500 μM DNM0195 | 0.39 μg/mL |
| Gentamicin | 0.39 μg/mL |
| Gentamicin + 500 μM DNM0195 | 0.195 μg/mL |

### Example 26

### Toxicity Assessment of DNM0195 in Cell Culture

Chinese hamster ovary cells (CHO-K1) were seeded in LabTek 8-well chamber slides in 0.3 ml DMEM+10% FBS and cultured for 24 h at 37° C., 5% CO₂. Medium was removed and DNM0195 at 1.0 or 0.1 mM in ethanol was added in fresh medium (final ethanol concentration 2% v/v). After 24 h further incubation, cells were stained with crystal violet and photographed under a microscope. Healthy stained cells were abundant in controls (±2% carrier ethanol) and in the presence of 0.1 mM and 1 mM of DNM0195.

### Example 27

In vivo Acute Toxicology Assessment of DNM0195

A suspension of DNM0195 was administered interperitoneally to mice under controlled conditions. The weight of each mouse was determined prior to administration. The weight range measured was 21.0 g to 24.5 g. Four mice were given 30 mg/kg, eight were given 100 mg/kg, and 4 were given 300 mg/kg, for a total of sixteen mice. Each mouse was observed at 30 min and 4 h for behavioral anomalies and submitted to a rotorod ataxia test. None of the animals showed any harmful effects. This procedure was repeated for a cohort of eight mice at 2,000 mg/kg with similar results. Liver biopsies were normal.

### Results

As the above results indicate, a subset of these compounds has potent pro-antibiotic activity and low toxicity in vitro and in vivo. Such compounds may be of significant benefit for the amelioration of sick conditions of infection and/or post-infective inflammatory disease.

### Equivalents

Those skilled in the art will recognize, or be able to ascertain using no more than routine experimentation, many equivalents to the specific embodiments of the invention described herein. Such equivalents are intended to be encompassed by the following claims.

We claim:

1. A compounds or pharmaceutically acceptable salt thereof comprising the structure of Formula I, 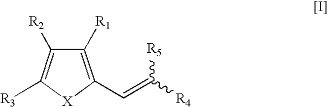
wherein
X is O, S, NH, or N-alkyl;
R₁ and R₂ are each, independently, hydrogen, tetrazole, alkylcarboxytetrazole, or alkyltetrazole (with the proviso that if R₁ is not hydrogen, R₂ must be, and vice versa);
R₃ is a halogen, alkyl halogen, 1,2-diphenylethyl, m-acrylylphenyl, substituted or unsubstituted N-phenylcarboxamido, substituted or unsubstituted alkyl, substituted or unsubstituted aryl or arylalkyl or substituted or unsubstituted heteroaryl or heteroarylalkyl;
R₄ is carboxylic acid or its alkyl esters, or a bioisosteric equivalent thereof, where R₄ may be connected cis or trans to the double bond and R₅ will necessarily be in the cis/trans position opposite to that of R₄; and
R₅ is hydrogen, or carboxylic acid or its alkyl esters, or a bioisosteric equivalent thereof.

2. The compound of claim 1 wherein X is S; R₁ and R₂ are each, independently, hydrogen, tetrazole, 1-methyltetrazole, or 2-methyl tetrazole (with the proviso that if R₁ is not hydrogen, R₂ must be, and vice versa); R₄ is carboxylic acid; R₅ is hydrogen; and R₃ is selected from the group consisting of: substituted or unsubstituted benzyl; substituted or unsubstituted phenyl; substituted or unsubstituted biphenyl; substituted or unsubstituted phenylbenzyl; or substituted or unsubstituted naphthyl.

3. A compound comprising the structure of Formula II, 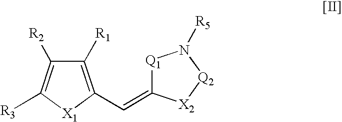
wherein
X₁ is O, S, NH, or N-alkyl;
X₂ is C, O, S, NH, or N-alkyl;
R₁ and R₂ are each, independently, hydrogen, tetrazole, or alkyltetrazole (with the proviso that if R₁ is not hydrogen, R₂ must be, and vice versa);
R₃ is a halogen, alkylhalogen, substituted or unsubstituted N-phenylcarboxyamido, substituted or unsubstituted alkyl, substituted or unsubstituted aryl or arylalkyl or substituted or unsubstituted heteroaryl or heteroarylalkyl;
R₅ is H or alkyl; and
Q₁ and Q₂ are each, independently, C or C(O) (if X₂ and Q₂ are both C, the bond between them may be single or double in order);
or a pharmaceutically acceptable salts thereof.

4. A compound according to any one of claim 1, 2, or 3, wherein alkyl refers to substituted or unsubstituted normal, branched, or cycle C₁-C₆ alkyl groups; aryl refers to substituted or unsubstituted phenyl or naphthyl; heteroaryl refers to substituted or unsubstituted indolyl, imidazolyl, or pyridyl; arylalkyl refers to substituted or unsubstituted benzyl, naphthylmethyl, or biphenylmethyl; and heteroarylalkyl refers to substituted or unsubstituted indolylmethyl, imidazolylmethyl, or pyridylmethyl.

5. A compound of claim 4, wherein alkyl refers to normal or branched C₁-C₄ alkyl groups.

6. A compound selected from the group consisting of (Z)-methyl 3-(5-benzyl-4-(1H-tetrazol-5-yl)thiophen-2-yl)acrylate, (E)-methyl 3-(5-benzyl-4-(1H-tetrazol-5-yl)thiophen-2-yl)acrylate, (Z)-3-(5-benzyl-4-(1H-tetrazol-5-yl)thiophen-2-yl)acrylic acid, (E)-3-(5-benzyl-4-(1H-tetrazol-5-yl)thiophen-2-yl)acrylic acid, (E)-methyl 3-(5-benzyl-4-(1-methyl-1H-tetrazol-5-yl)thiophen-2-yl)acrylate, (E)-methyl 3-(5-benzyl-4-(2-methyl-2H-tetrazol-5-yl)thiophen-2-yl)acrylate, (E)-3-(5-benzyl-4-(2-methyl-2H-tetrazol-5-yl)thiophen-2-yl)acrylic acid, (E)-3-(5-benzyl-4-(1-methyl-1H-tetrazol-5-yl)thiophen-2-yl)acrylic acid, (E)-3-(5-(4-fluorophenyl)-4-(1H-tetrazol-5-yl)thiophen-2-yl)acrylic acid, (E)-3-(5-(4-biphenylyl)-4-(1H-tetrazol-5-yl)thiophen-2-yl)acrylic acid, (E)-methyl 3-(5-(naphthalen-1-yl)-4-(1H-tetrazol-5-yl)thiophen-2-yl)acrylate, (E)-3-(5-(naphthalen-1-yl)-4-(1H-tetrazol-5-yl)thiophen-2-yl)acrylic acid, (E)-methyl 3-(5-(4-fluorophenyl)-3-(1H-tetrazol-5-yl)thiophen-2-yl)acrylate, (E)-3-(5-(4-fluorophenyl)-3-(1H-tetrazol-5-yl)thiophen-2-yl)acrylic acid, (E)-methyl 3-(5-(4-fluorophenyl)-3-(2-methyl-2H-tetrazol-5-yl)thiophen-2-yl)acrylate, (E)-3-(5-(4-fluorophenyl)-3-(2-methyl-2H-tetrazol-5-yl)thiophen-2-yl)acrylic acid, (E)-3-(5-(4-fluorophenyl)-3-(1-methyl-1H-tetrazol-5-yl)thiophen-2-yl)acrylic acid, (E)-methyl 3-(4-(2-methyl-2H-tetrazol-5-yl)-5-(naphthalen-1-yl)thiophen-2-yl)acrylate, (E)-methyl 3-(5-(4-biphenyl)-3-(1H-tetrazol-5-yl)thiophen-2-yl)acrylate, (E)-3-(5-(biphenyl-4-yl)-3-(1H-tetrazol-5-yl)thiophen-2-yl)acrylic acid, (E)-methyl 3-(5-benzyl-3-(1H-tetrazol-5-yl)thiophen-2-yl)acrylate, 5-((5-benzyl-3-(1H-tetrazol-5-yl)thiophen-2-yl)methylene)thiazolidine-2,4-dione, 2-((5-benzyl-4-(1H-tetrazol-5-yl)thiophen-2-yl)methylene)malonic acid, (E)-3-(5-benzyl-4-(2-(2-carboxyethyl)-2H-tetrazol-5-yl)thiophen-2-yl)acrylic acid, (E)-3-(3-((5-((E)-2-carboxyvinyl)-3-(1H-tetrazol-5-yl)thiophen-2-yl)methyl)phenyl)acrylic acid.

7. A method of treating a subject having an infection, comprising administering to the subject having an infection a therapeutically effective amount of a compound according to any one of claims 1-6.

8. A method of treating a subject having an infection, comprising administering to the subject in need of such treatment a therapeutically effective amount a compound according to any one of claims 1-6 in combination with an antibiotic agent.

9. A method of treating a subject having an inflammatory disease, comprising administering to the subject having the inflammatory disease a therapeutically effective amount of at least one compound according to any one of claims 1-6.

10. A method of treating a subject having an inflammatory disease, comprising administering to the subject having the inflammatory disease a therapeutically effective amount of at least one compound according to any one of claims 1-6 in combination with an antibiotic agent.

11. The method of claim 7 or 8, wherein said infection is selected from the group consisting of blood-stream infection, brucellosis, campylobacteriosis, Cat Scratch fever, cholera, gonorrhea, legionellosis, leptospirosis, Lyme disease, melioidosis, meningitis, pertussis, plague, pneumonia, salmonellosis, shigellosis, syphilis, tularemia, typhoid fever, and urinary tract infection.

12. The method of claim 9 or 10, wherein said inflammatory disease is septic shock or systemic inflammatory response syndrome.

13. The method of any one of claims 7-12 wherein said subject is human.

14. The method of any one of claims 7-12 wherein said compound is administered to said subject using a pharmaceutically acceptable formulation.

15. The method of any one of claims 7-12 wherein said compound is administered to the subject orally.

16. The method of any one of claims 7-12 wherein said compound is administered to the subject intravenously.

17. The method of any one of claims 7-12 wherein said compound is administered to the subject intraperitoneally.
